SEQUENCE LISTING (40 sequences)

SEQ ID NO: 1 - DNA, 1356 bp, Artificial Sequence
  H2B6Hc-3Fc0088 (Heavy Chain)
caggttcagctggtgcagtctggagctgaggtgaagaagcctggggcctcagtgaaggtctcctgcaaggcttctggttacacctttaccaactactggatacactgggtgcgacaggcccctggacaagggcttgagtggattggagtgattgatccttctgatacttatccaaattacaataaaaagttcaagggcagagtcaccatgaccgtagacacatccacgagcacagcctacatggagctgaggagcctgagatctgacgacacggccgtgtattactgtgcgagaaacggtgattccgattattactctggtatggactactgggggcaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttacccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgccggaggagcagtacaacagcacgctccgtgtggtcagcatcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctctcgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - DNA, 645 bp, Artificial Sequence
  H2B6 Lc-5 (Light chain)
gaaattgtgctgactcagtctccagactttcagtctgtgactccaaaggagaaagtcaccttcacctgcaggaccagtcagagcattggcacaaacatacactggtaccagcagaaaccagatcagtctccaaagctcctcatcaaggaggtttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 3 - protein, 451 aa, Artificial Sequence
  H2B6Hc-3Fc0088 (Heavy Chain)
QVQLVQSGAEVKKPGASVKVSCKASGYTFTNYWIHWVRQAPGQGLEWIGVIDPSDTYPNYNKKFKGRVTMTVDTSTSTAYMELRSLRSDDTAVYYCARNGDSDYYSGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 4 - protein, 214 aa, Artificial Sequence
  H2B6 Lc-5 (Light chain)
EIVLTQSPDFQSVTPKEKVTFTCRTSQSIGTNIHWYQQKPDQSPKLLIKEVSESISGVPSRFSGSGSGTDFTLTINSLEAEDAATYYCQQSNTWPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 121 aa, Artificial sequence
  Humanized heavy chain variable region
QVQLVQSGAEVKKPGASVKVSCKASGYTFTNYWIHWVRQAPGQGLEWMGVIDPSDTYPNYNKKFKGRVTMTTDTSTSTAYMELRSLRSDDTAVYYCARNGDSDYYSGMDYWGQGTTVTVSS

SEQ ID NO: 6 - protein, 121 aa, Artificial Sequence
  H2B6VH-A
QVQLVQSGAEVKKPGASVKVSCKASGYTFTNYWIHWVRQAPGQGLEWIGVIDPSDTYPNYNKKFKGRVTMTVVVSTSTAYMELRSLRSDDTAVYYCARNGDSDYYSGMDYWGQGTTVTVSS

SEQ ID NO: 7 - protein, 121 aa, Artificial Sequence
  H2B6VH-3
QVQLVQSGAEVKKPGASVKVSCKASGYTFTNYWIHWVRQAPGQGLEWIGVIDPSDTYPNYNKKFKGRVTMTVDTSTSTAYMELRSLRSDDTAVYYCARNGDSDYYSGMDYWGQGTTVTVSS

SEQ ID NO: 8 - protein, 107 aa, Artificial sequence
  Humanized 2B6 light chain variable region - Hu2B6VL-1
EIVLTQSPDFQSVTPKEKVTITCRTSQSIGTNIHWYQQKPDQSPKLLIKNVSESISGVPSRFSGSGSGTDFTLTINSLEAEDAATYYCQQSNTWPFTFGGGTKVEIK

SEQ ID NO: 9 - protein, 107 aa, Artificial sequence
  Humanized 2B6 light chain variable region - Hu2B6VL-2
EIVLTQSPDFQSVTPKEKVTITCRTSQSIGTNIHWYQQKPDQSPKLLIKYVSESISGVPSRFSGSGSGTDFTLTINSLEAEDAATYYCQQSNTWPFTFGGGTKVEIK

SEQ ID NO: 10 - protein, 107 aa, Artificial sequence
  Humanized 2B6 light chain variable region - Hu2B6VL-3
EIVLTQSPDFQSVTPKEKVTITCRTSQSIGTNIHWYQQKPDQSPKLLIKYASESISGVPSRFSGSGSGTDFTLTINSLEAEDAATYYCQQSNTWPFTFGGGTKVEIK

SEQ ID NO: 11 - protein, 107 aa, Artificial Sequence
  H2B6VL-5
EIVLTQSPDFQSVTPKEKVTFTCRTSQSIGTNIHWYQQKPDQSPKLLIKEVSESISGVPSRFSGSGSGTDFTLTINSLEAEDAATYYCQQSNTWPFTFGGGTKVEIK

SEQ ID NO: 12 - protein, 115 aa, mus sp.
  mouse 3H7 Heavy Chain Variable Region
EVKFEESGGGLVQPGGSMKLSCAASGFTFSDAWMDWVRQGPEKGLEWVAEIRNKANNLATYYAESVKGRFTIPRDDSKSSVYLHMNSLRAEDTGIYYCYSPFAYWGQGTLVTVSA

SEQ ID NO: 13 - protein, 107 aa, mus sp.
  mouse 3H7 Light Chain Variable Region
DIQMTQSPSSLSASLGERVSLTCRASQEISGYLSWLQQKPDGTIRRLIYAASTLDSGVPKRFSGSWSGSDYSLTISSLESEDFADYYCLQYVSYPYTFGGGTKLEIK

SEQ ID NO: 14 - protein, 5 aa, Artificial sequence
  2B6 Heavy chain variable region - CDR1
NYWIH

SEQ ID NO: 15 - protein, 5 aa, Artificial Sequence
  3H7 Heavy Chian Variable region - CDR1
DAWMD

SEQ ID NO: 16 - protein, 17 aa, Artificial sequence
  2B6 Heavy chain variable region - CDR2
VIDPSDTYPNYNKKFKG

SEQ ID NO: 17 - protein, 19 aa, Artificial Sequence
  3H7 Heavy Chian Variable region - CDR2
EIRNKANNLATYYAESVKG

SEQ ID NO: 18 - protein, 12 aa, Artificial sequence
  2B6 Heavy chain variable region - CDR3
NGDSDYYSGMDY

SEQ ID NO: 19 - protein, 6 aa, Artificial Sequence
  3H7 Heavy Chian Variable region - CDR3
YSPFAY

SEQ ID NO: 20 - protein, 11 aa, Artificial sequence
  2B6 Light chain variable region - CDR1
RTSQSIGTNIH

SEQ ID NO: 21 - protein, 11 aa, Artificial Sequence
  3H7 Light Chian Variable region - CDR1
RASQEISGYLS

SEQ ID NO: 22 - protein, 7 aa, Artificial sequence
  2B6 Light chain variable region - CDR2
NVSESIS

SEQ ID NO: 23 - protein, 7 aa, Artificial sequence
  2B6 Light chain variable region - CDR2
YVSESIS

SEQ ID NO: 24 - protein, 7 aa, Artificial sequence
  2B6 Light chain variable region - CDR2
YASESIS

SEQ ID NO: 25 - protein, 7 aa, Artificial Sequence
  3H7 Light Chian Variable region - CDR2
AASTLDS

SEQ ID NO: 26 - protein, 9 aa, Artificial sequence
  2B6 Light chain variable region - CDR3
QQSNTWPFT

SEQ ID NO: 27 - protein, 9 aa, Artificial Sequence
  3H7 Light Chian Variable region - CDR3
LQYVSYPYT

SEQ ID NO: 28 - protein, 451 aa, Artificial Sequence
  H2B6Hc-AFc0088 (Heavy Chain)
QVQLVQSGAEVKKPGASVKVSCKASGYTFTNYWIHWVRQAPGQGLEWIGVIDPSDTYPNYNKKFKGRVTMTVVVSTSTAYMELRSLRSDDTAVYYCARNGDSDYYSGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 29 - DNA, 363 bp, Artificial Sequence
  H2B6VH-3
caggttcagctggtgcagtctggagctgaggtgaagaagcctggggcctcagtgaaggtctcctgcaaggcttctggttacacctttaccaactactggatacactgggtgcgacaggcccctggacaagggcttgagtggattggagtgattgatccttctgatacttatccaaattacaataaaaagttcaagggcagagtcaccatgaccgtagacacatccacgagcacagcctacatggagctgaggagcctgagatctgacgacacggccgtgtattactgtgcgagaaacggtgattccgattattactctggtatggactactgggggcaagggaccacggtcaccgtctcctca

SEQ ID NO: 30 - DNA, 321 bp, Artificial Sequence
  H2B6VL-5
gaaattgtgctgactcagtctccagactttcagtctgtgactccaaaggagaaagtcaccttcacctgcaggaccagtcagagcattggcacaaacatacactggtaccagcagaaaccagatcagtctccaaagctcctcatcaaggaggtttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaa

SEQ ID NO: 31 - DNA, 321 bp, Artificial sequence
  Humanized 2B6 light chain variable region - Hu2B6VL-1
gaaattgtgctgactcagtctccagactttcagtctgtgactccaaaggagaaagtcaccatcacctgcaggaccagtcagagcattggcacaaacatacactggtaccagcagaaaccagatcagtctccaaagctcctcatcaagaatgtttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaa

SEQ ID NO: 32 - DNA, 321 bp, Artificial sequence
  Humanized 2B6 light chain variable region - Hu2B6VL-2
gaaattgtgctgactcagtctccagactttcagtctgtgactccaaaggagaaagtcaccatcacctgcaggaccagtcagagcattggcacaaacatacactggtaccagcagaaaccagatcagtctccaaagctcctcatcaagtatgtttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaa

SEQ ID NO: 33 - DNA, 321 bp, Artificial sequence
  Humanized 2B6 light chain variable region - Hu2B6VL-3
gaaattgtgctgactcagtctccagactttcagtctgtgactccaaaggagaaagtcaccatcacctgcaggaccagtcagagcattggcacaaacatacactggtaccagcagaaaccagatcagtctccaaagctcctcatcaagtatgcttctgagtctatctctggagtcccatcgaggttcagtggcagtggatctgggacagatttcaccctcaccatcaatagcctggaagctgaagatgctgcaacgtattactgtcaacaaagtaatacctggccgttcacgttcggcggagggaccaaggtggagatcaaa

SEQ ID NO: 34 - DNA, 363 bp, Artificial sequence
  Humanized heavy chain variable region - Hu2B6VH-1
caggttcagctggtgcagtctggagctgaggtgaagaagcctggggcctcagtgaaggtctcctgcaaggcttctggttacacctttaccaactactggatacactgggtgcgacaggcccctggacaagggcttgagtggatgggagtgattgatccttctgatacttatccaaattacaataaaaagttcaagggcagagtcaccatgaccacagacacatccacgagcacagcctacatggagctgaggagcctgagatctgacgacacggccgtgtattactgtgcgagaaacggtgattccgattattactctggtatggactactgggggcaagggaccacggtcaccgtctcctca

SEQ ID NO: 35 - DNA, 345 bp, mus sp.
  mouse 3H7 Heavy Chain Variable Region
gaagtgaagtttgaggagtctggaggaggcttggtgcaacctggaggatccatgaaactctcttgtgctgcctctggattcacttttagtgacgcctggatggactgggtccgccagggtccagagaaggggcttgagtgggttgctgaaattagaaacaaagctaataatcttgcaacatactatgctgagtctgtgaaagggaggttcaccatcccaagagatgattccaaaagtagtgtctacctgcacatgaacagcttaagagctgaagacactggcatttattactgttatagtccctttgcttactggggccaagggactctggtcactgtctctgca

SEQ ID NO: 36 - DNA, 321 bp, mus sp.
  mouse 3H7 Light Chain Variable Region
gacatccagatgacccagtctccatcctccttatctgcctctctgggagaaagagtcagtctcacttgtcgggcaagtcaggaaattagtggttacttaagctggcttcagcagaaaccagatggaactattagacgcctgatctacgccgcatccactttagattctggtgtcccaaaaaggttcagtggcagttggtctgggtcagattattctctcaccatcagcagccttgagtctgaagattttgcagactattactgtctacaatatgttagttatccgtatacgttcggaggggggaccaagctggaaataaaa

SEQ ID NO: 37 - DNA, 25 bp, Artificial Sequence
  Forward primer used to amplify CD32B message
cctcacctggagttccaggagggag

SEQ ID NO: 38 - DNA, 21 bp, Artificial Sequence
  Reverse primer used to amplify CD32B message
aactttgtcagcctcatcagg

SEQ ID NO: 39 - DNA, 24 bp, Artificial Sequence
  forward primer used to amplify GADPH message
ttcgacagtcagccgcatcttctt

SEQ ID NO: 40 - DNA, 24 bp, Artificial Sequence
  reverse primer used to amplify GADPH message
gcccaatacgaccaaatccgttga

(19) United States
(12) Patent Application Publication
(10) Pub. No.: US 2008/0317745 A1
(43) Pub. Date: Dec. 25, 2008

(54) Methods of diagnosing, treating, or preventing plasma cell disorders

(76) Inventors: Adam M. Boruchov, New York, NY (US); Raymond L. Comenzo, New York, NY (US)

Correspondence Address: THE NATH LAW GROUP, 112 South West Street, Alexandria, VA, 22314, US

(21) Appl. No.: 11/898,932

(22) Filed: Sep. 17, 2007

Related U.S. Application Data
(60) Provisional application No. 60/845,472, filed on Sep. 15, 2006.

(51) Int. Cl. A61K 39/395; A61P 35/02; A61P 7/00; A61K 39/44; C12Q 1/02; G01N 33/566

(52) U.S. Cl. 424/133.1; 424/173.1; 424/153.1; 424/178.1; 435/29; 435/7.21

(57) ABSTRACT

The present invention relates to methods and compositions for the diagnosis, treatment, management, or prevention of plasma cell disorders, including systemic light-chain amyloidosis (AL) and multiple myeloma (MM). In particular, the invention encompasses the use of anti-CD32B antibodies, analogs, derivatives or fragments thereof, or compounds or agents that bind to CD32B and modulate CD32B activity in the plasma cells of mammals. The invention also encompasses the use of anti-CD32B antibodies, analogs, derivatives or fragments thereof, or CD32B binding compounds or agents in combination with or in addition to other cancer therapies for the treatment, prevention, management, or amelioration of a plasma cell disorder characterized by the expression of CD32B, or one or more symptoms thereof. The invention further relates to the use of anti-CD32B antibodies, analogs, derivatives or fragments thereof for the detection of aberrant or altered expression of CD32B in plasma cells, to diagnosis and/or characterize a plasma cell disorder.

This application claims benefit of U.S. Provisional Patent Application No. 60/845,472, filed Sep. 15, 2006, the entire contents of which is hereby incorporated by reference herein.

This invention was made in part with government support under grant number NIH K08 A1061313-02 awarded by the U.S. National Institutes of Health. The United States Government may therefore have certain rights in the invention.

### 1. FIELD OF THE INVENTION

The present invention relates to methods and compositions for the diagnosis, treatment, management, or prevention of plasma cell disorders, including systemic light-chain amyloidosis (AL) and multiple myeloma (MM). In particular, the invention encompasses the use of anti-CD32B antibodies, analogs, derivatives or fragments thereof, or compounds or agents that bind to CD32B and modulate CD32B activity in the plasma cells of mammals. The invention also encompasses the use of anti-CD32B antibodies, analogs, derivatives or fragments thereof, or CD32B binding compounds or agents in combination with or in addition to other cancer therapies for the treatment, prevention, management, or amelioration of a plasma cell disorder characterized by the expression of CD32B, or one or more symptoms thereof. The invention further relates to the use of anti-CD32B antibodies, analogs, derivatives or fragments thereof for the detection of aberrant or altered expression of CD32B in plasma cells, to diagnosis and/or characterize a plasma cell disorder.

### 2. BACKGROUND OF THE INVENTION

2.1 Plasma Cell Disorders

Plasma cells disorders result from the uncontrolled division or multiplication of a plasma cell clone. Plasma cells arise from activated B lymphocytes (i.e., B cells). Each B cell produces a unique receptor, known as the B cell receptor, arrayed on its cell surface that is specific for a foreign substance, i.e., antigen. When a B cell receptor binds its cognate antigen, the cell expressing the receptor is activated to re-enter the cell cycle, producing many clonal copies of itself. The clones mature into plasma cells that reside principally in the bone marrow and that are specialized to produce copies of the B cell receptor that are released into the blood stream as antibodies.

In a plasma cell disorder, the plasma cell or the parent B cell suffers genetic damage resulting in suppression of or insensitivity to the normal restraints on cell division and/or activity. Daughter plasma cells derived from such cells are malignant in that they may divide unchecked and/or generate excess amount of the same immunoglobulin (antibody). Often the immunoglobulin produced is incomplete or has an incorrect conformation that can result in accumulation of the protein (also known as monoclonal protein, M protein, paraprotein or amyloid protein, dependent on the specific disorder) in the serum, tissues or organs (especially the kidneys), leading to organ dysfunction and/or failure.

Plasma cell disorders include monoclonal gammopathies of undetermined significance (MGUS), multiple myeloma (MM), macroglobulinemia, heavy chain diseases, and systemic light-chain amyloidosis (AL), which are differentiated based on the proliferative nature of the clone, the extent of marrow involvement, and the type of M protein expressed. Although new immunotherapies such as rituximab and alemtuzumab have improved disease-free and overall survival in some B-cell malignancies, such therapies have not proven effective in the treatment of plasma cell disorders in part because the target antigens, CD20 and CD52, respectively, are not sufficiently expressed by the malignant clonal plasma cells. Thus, there is a need for identification and development of improved therapy of plasma cell disorders.

2.1.1 Multiple Myeloma

Multiple myeloma (MM) is the most common form of plasma cell malignancy, causing an estimate of more than 11,000 deaths and over 16,000 new malignant diagnoses in 2006 (American Cancer Society, Cancer Facts and Figures 2006; available at http://www.cancer.org/downloads/STT/CAFF2006PWSecured.pdf). It is highly treatable but rarely curable, with prognosis for survival after diagnosis ranging from days to greater than 10 years. Epidemiological studies have shown that at least one third of MM emerge from pre-existing benign plasma cell disorders, i.e., MGUS, suggesting that at least two types of MM exist: MM secondary to preexisting MGUS and primary MM (i.e., de novo MM that bypasses the MGUS stage) (Kyle, et al., 1994, J. Clin. Oncol. 12:1577-1583, which is hereby incorporated by reference in its entirety). Finally, some subjects present with advanced, aggressive MM at diagnosis, termed primary plasma cell leukemia (PCL). The etiology of the disease is unknown, but the presence in families of both MM and MGUS suggests a genetic link for the diseases. The stage of the disease at presentation is a strong determinant of survival, but it has little influence on the choice of therapy since almost all subjects, except for rare subjects with solitary bone tumors or extramedullary plasmacytomas, have generalized disease.

The clinical manifestations of MM vary, but may include tumor formation, osteolysis, hemopoiesis, anemia, recurrent bacterial infections and renal disease. MM tumors usually develop in areas of hemopoietically active marrow, increasing myeloma load at the expense of other cells in the marrow. Additionally, the aberrant production of cytokines by many MM cells/tumors can lead to a disruption of the bone remodeling mechanisms at the tumor site. MM may thus result in decreased marrow function (e.g., anemia and decreased immune function) and/or, bone resorption (osteolytic lesions) in areas surrounding the tumor (see, e.g., Mulleman et al., 2004, Joint Bone Spine, 71:79-83, hereby incorporated by reference in its entirety). Additionally, the malignant cells normally secrete abnormal amounts of monoclonal immunoglobulin (M protein), which may be IgG, IgA, IgD, or IgE or a fragment thereof, or κ or λ light chains (also known as Bence Jones protein). In 0.5% of MM subjects, two or more M proteins are detected (Pruzanski and Ogryzlo, 1970, Adv. Clin. Chemo. 13:355, hereby incorporated by reference in its entirety); rarely, MM presents with no detectable secretion of M protein.

Treatment of MM is influenced by the age and general health of the subject, prior therapy, the stage of the disease and the presence of complications of the disease. Standard treatments for multiple myeloma correspond to those associated with similar malignancies, and include: biophosphonates; chemotherapy (e.g., alkylating agents, vincristine, doxorubicin); autologous stem cell transplantation; thalidomide; and corticosteroids (e.g., prednisone and dexamethasone). Corticosteroids have been shown to suppress tumor cell proliferation and to reduce M protein in up to 60% of previously untreated subjects, and in 20% to 40% of subjects refractive to other therapies. Dexamethasone is also used to protect against renal damage induced by the light chains. However, the side effects of long-term dexamethasone administration are of serious concern. In particular, chronic dexamethasone treatment can cause osteoporosis, thus reducing its utility in myeloma therapy because a major sequela of myeloma is bone resorption. Thus there is a need to obtain an improvement over existing therapies for multiple myeloma.

2.1.2 Systemic Light Chain Amyloidosis

In contrast to MM, the malignant plasma cells associated with systemic light-chain amyloidosis (AL) are generally not present in large numbers and are usually not proliferative. Biologic aggression in AL is a result of organ failure and debilitation due to light-chain toxicity and fibrillar deposition disease rather than the increasing myeloma burden. Although a minority of AL subjects are able to achieve long-term hematologic remissions (>8 years), many die of the disease within 3 years of diagnosis.

The abnormal proteins secreted by the malignant AL cells form insoluble (amyloid) protein fibrils that accumulate in various tissues and organs throughout the body, (e.g., in the kidneys, spleen, heart, liver, adrenals, skeletal muscle, skin, ligaments, and/or gastrointestinal tract) resulting in heterogeneous presentations. For example, subjects may present with macroglossia (e.g., problems speaking or swallowing); carpal tunnel syndrome with median nerve compression; or peripheral neuropathy with sensory disturbances, weakness or autonomic nerve dysfunction. Amyloid may infiltrate regions of the gastrointestinal tract causing obstruction, hemorrhage, malabsorption or disturbances in intestinal motility. Cardiac involvement can result in stiff, poorly contractile muscle tissue presenting as cardiomegaly or resulting in congestive heart failure. Amyloidosis can also present as a coagulopathy associated with factor X deficiency due to the selective binding of factor X to amyloid.

The resistance of amyloid to phagocytosis or proteolysis limits removal of the protein deposits by host defense mechanisms, resulting in a progressive disease ultimately causing death by destruction of involved tissues. Because the deposition of amyloid far exceeds the host clearance ability, treatment of the disease optimally involves complete cure or prolonged remission of the underlying plasma cell disorder and/or malignancy. In some AL subjects, durable, long-term remission is effected by high-dose melphalan and stem cell transplant (Adams and Weiner, 2005, Nat. Biotechnol. 23:1147-1157, hereby incorporated by reference in its entirety). However, the data indicate that the key to treating AL is to effectively target the malignant clonal plasma cell.

2.2 Cancer Therapy

Currently, therapy for malignant disorders, e.g., cancer, may involve surgery, chemotherapy, hormonal therapy and/or radiation treatment to eradicate neoplastic cells in a subject (See, for example, Stockdale, 1998, “Principles of Cancer Subject Management”, in Scientific American Medicine, vol. 3, Rubenstein and Federman, eds., Chapter 12, Section IV). Recently, such therapies could also involve biological therapy or immunotherapy. All of these approaches pose significant drawbacks for the subject. Surgery, for example, may be contraindicated due to the health of the subject or may be unacceptable to the subject. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although can be effective, is often used to prevent or delay recurrence of cancer after other treatments have removed the majority of the malignant and/or cancer cells. Biological therapies/immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of plasma cell disorders and/or cancer. A significant majority of chemotherapeutics act by inhibiting DNA synthesis, either directly, or indirectly by inhibiting the biosynthesis of the deoxyribonucleotide triphosphate precursors, to prevent DNA replication and concomitant cell division (See, for example, Gilman et al., Goodman and Gilman's: The Pharmacological Basis of Therapeutics, Eighth Ed. (Pergamom Press, New York, 1990)). These agents, which include alkylating agents, such as nitrosourea, melphalan, and carmustine; anti-metabolites, such as methotrexate and hydroxyurea; and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (See, for example, Stockdale, 1998, “Principles Of Cancer Subject Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

Plasma cell disorders are generally treated with single agent chemotherapy, combination chemotherapy and, where localized disease can be ascertained, radiation therapy. These treatments can reduce morbidity and/or improve survival, albeit they carry significant side effects. The response of plasma cell disorders to the various forms of treatment is mixed. Prognosis for plasma cell malignancies can be quite variable, with some subjects achieving remission for greater than 10 years. Certain subjects, however, fail to respond and disease recurrence with resistance to treatment ensues with time, particularly with the most aggressive variants of the disease.

Investigational therapies for the treatment of refractory plasma cell malignancies include autologous and allogeneic bone marrow or stem cell transplantation and gene therapies. Recently, immunotherapy using monoclonal antibodies to target myeloma associated antigens has been introduced in the treatment of MM. The use of monoclonal antibodies to direct radionuclides, toxins, or other therapeutic agents offers the possibility that such agents can be delivered to plasma cells directly or to plasma cell tumor sites, preferably specifically, thus limiting toxicity to normal tissues.

There is a significant need for alternative treatments of plasma cell malignancies, particularly for treatment of malignancies that have proved refractory to standard cancer treatments, such as surgery, radiation therapy, chemotherapy, and hormonal therapy. A promising alternative is immunotherapy, in which cancer cells are specifically targeted by cancer antigen-specific antibodies. Major efforts have been directed at harnessing the specificity of the immune response, for example, hybridoma technology has enabled the development of tumor selective monoclonal antibodies (See Green M. C. et al., 2000 Cancer Treat Rev., 26: 269-286; Weiner LM, 1999 Semin. Oncol. 26(suppl. 14):43-51), and in the past few years, the Food and Drug Administration has approved the first MAbs for cancer therapy. Interest in immunotherapy for the treatment of plasma cell disorders has focused on the treatment of myeloma, and in particular, the targeting of precursor cells (B-cells) and/or the identification of MM-specific antigens. However, no clinical trials have been reported describing a response to monoclonal antibody in plasma cell dyscrasias (Treon et al., 1999, Sem. Oncol. S14:97-106, herein incorporated by reference in its entirety).

There is a need for alternative therapies for plasma cell disorders and/or malignancies, especially for subjects that are refractory for standard cancer treatments and/or new immunotherapies.

2.3 FcγRII (CD32)

FcγRII (CD32) proteins are 40 KDa integral membrane glycoproteins which bind only the complexed IgG due to a low affinity for monomeric Ig (10⁶ M⁻¹). This receptor is the most widely expressed FcγR, present on all hematopoietic cells, including monocytes, macrophages, B cells, NK cells, neutrophils, mast cells, and platelets. FcγRII has only two immunoglobulin-like regions in its immunoglobulin binding chain and hence a much lower affinity for IgG than FcγRI. There are three human FcγRII genes (FcγRII-A, also known as CD32A, FcγRII-B, also known as CD32B, and FcγRII-C, also known as CD32C), all of which bind IgG in aggregates or immune complexes.

Distinct differences within the cytoplasmic domains of FcγRII-A (CD32A) and FcγRII-B (CD32B) create two functionally heterogeneous responses to receptor ligation. The fundamental difference is that the A isoform initiates intracellular signaling leading to cell activation such as phagocytosis and respiratory burst, whereas the B isoform initiates inhibitory signals, e.g., inhibiting B-cell activation.

Both activating and inhibitory signals are transduced through the FcγRs following ligation; however, two distinct domains within the cytoplasmic signaling domains of the receptor called immunoreceptor tyrosine based activation motifs (ITAMS) or immunoreceptor tyrosine based inhibitory motifs (ITIMS) account for the different responses. The recruitment of different cytoplasmic enzymes to these structures dictates the outcome of the FcγR-mediated cellular responses. ITAM-containing FcγR complexes include FcγRI (CD64), FcγRIIA (CD32A), FcγRIIIA (CD16A), whereas ITIM-containing complexes only include FcγRIIB (CD32B).

Human neutrophils express the FcγRIIA (CD16A) gene. FcγRIIA (CD16A) clustering via immune complexes or specific antibody cross-linking serves to aggregate ITAMs along with receptor-associated kinases which facilitate ITAM phosphorylation. ITAM phosphorylation serves as a docking site for Syk kinase, activation of which results in activation of downstream substrates (e.g., PI₃K). Cellular activation leads to release of proinflammatory mediators.

The FcγRIIB (CD32B) gene is expressed on B lymphocytes; its extracellular domain is 96% identical to FcγRIIA (CD32A) and binds IgG complexes in an indistinguishable manner. The presence of an ITIM in the cytoplasmic domain of FcγRIIB (CD32B) defines this inhibitory subclass of FcγR. Recently the molecular basis of this inhibition was established. When co-ligated along with an activating FcγR, the ITIM in FcγRIIB (CD32B) becomes phosphorylated and attracts the SH2 domain of the inositol polyphosphate 5′-phosphatase (SHIP), which hydrolyzes phophoinositol messengers released as a consequence of ITAM-containing FcγR— mediated tyrosine kinase activation, consequently preventing the influx of intracellular Ca⁺⁺. Thus, crosslinking of FcγRIIB (CD32B) dampens the activating response to FcγR ligation and inhibits cellular responsiveness. B cell activation, B cell proliferation and antibody secretion is thus aborted.

### 3. SUMMARY OF THE INVENTION

The present invention provides methods of diagnosing or assessing the aggressivity of a plasma cell disorder in a subject, particularly systemic light-chain amyloidosis (AL), multiple myeloma (MM), and plasma cell leukemia (PCL). In certain embodiments, said methods comprise characterizing the expression of CD32B on the plasma cells of a subject by detecting in a biological sample from said subject a population of plasma cells and determining the level of expression of CD32B by said population. CD32B is a B cell lineage surface antigen, discovered in the instant application to be present on the surface of plasma cell samples from AL subjects and a majority of the plasma cells from MM subjects. Thus, the expression profile makes it a suitable target for therapeutics for the treatment of certain plasma cell disorders. In addition, CD32B belongs to the category of inhibitory receptors, whose ligation delivers a negative signal. Therefore, antibodies and other molecules directed against CD32B could function to eliminate tumor cells by mechanisms that include complement dependent cytotoxicity (CDC), antibody-dependent cellular cytotoxicity (ADCC), and/or triggering an apoptotic signal. Such molecules could also be used to deliver toxic agents to diseased cells. In certain embodiments, the population of plasma cells is isolated from the biological sample prior to determining the level of CD32B expression. As discovered by the applicants, the reduction in expression of CD32B on plasma cells correlates with plasma cell disease and the aggressivity of the plasma cell disease. In certain embodiments, the level of CD32B expression is compared to that of a control cell population. In related embodiments, the control cell population is a population of non-diseased or non-malignant plasma cells from a healthy subject or other source of non-diseased cultured cells. In other embodiments, the control cell population is a non-diseased or non-malignant population of plasma cells from said subject. In certain embodiments, the CD32B expression of the plasma cells from said subject is less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2%, or less than 1% of the CD32B expression of the control cell population. In other embodiments, the CD32B expression of the plasma cell population from said subject is undetectable by standard methods known in the art for characterizing the expression of proteins on a cell surface, e.g., flow cytometry. In specific embodiments, the ratio of the level of CD32B expression of the plasma cells of said subject to the level of CD32B expression of the control cell population is inversely proportional to the aggressivity of said disease.

The methods of the invention provide a method for monitoring the progression of a plasma cell disorder (preferably systemic light-chain amyloidosis (AL), multiple myeloma (MM), or plasma cell leukemia (PCL)) in a subject comprising, step (a), detecting in a biological sample from said subject a population of plasma cells; step (b), determining the level of CD32B expression in said population; and step (c), at a subsequent time point, repeating steps (a) and (b). In specific embodiments, the level of CD32B expression determined in step (c) is compared with the level of CD32B expression determined in (b), or that determined at a prior time point, such that a change in the level is associated with the progression, regression or remission of said disorder. In some embodiments, said subject has received or is currently receiving cancer therapies during said monitoring. In related embodiments, the cancer therapy is changed based on the monitoring of change in CD32B expression of the plasma cells of said subject. In certain embodiments, a decrease in the level of CD32B expression is associated with progression of the disorder to a more aggressive disease state. In other embodiments, an increase in the level of CD32B expression is associated with a regression or remission of said disorder. In certain embodiments, step (c) is repeated at least once, at least twice, at least three times, at least four times, at least five times, at least six times, at least seven times, at eight times, at least nine times, at least ten times, at least twelve times, at least fourteen times, at least sixteen times, at least eighteen times, or at least twenty times or more. In certain embodiments, step (c) is performed 1 week, 2 weeks, 3 weeks, 4 weeks, 6 weeks, 8 weeks, 3 months, 4 months, 5 months, 6 months, 8 months, 10 months; 12 months, 14 months, 16 months, 18 months, 20 months, 22 months, or 24 months or longer after step (b). In other embodiments, step (c) may be repeated at 1 week, 2 week, 3 week, 4 week, 6 week, 8 week, 3 month, 4 month, 5 month, 6 month, 8 month, 10 month, 12 month, 14 month, 16 month, 18 month, 20 month, 22 month, or 24 month or longer intervals. In certain embodiments, step (a), (b) or (c) may be performed prior to administration of one or more cancer therapies. In other embodiments, step (a), (b), or (c) may be performed after administration of one or more cancer therapies. In still other embodiments, steps (a) and (b) may be performed prior to administration of one or more cancer therapies and step (c) performed subsequent thereto.

The invention encompasses methods comprising the use of a CD32 B binding agent. In preferred embodiments, the CD32B binding agent is an anti-CD32B antibody, analog, derivative, or fragment thereof. In preferred embodiments, said antibody, analog, derivative, or fragment thereof binds to CD32B with greater affinity than said antibody, analog, derivative, or fragment thereof binds to CD32A. In related embodiments, the CD32B binding agent of the invention, binds to CD32B with at least 2 times, at least 5 times, at least 10 times, at least 15 times, at least 20 times, at least 25 times, at least 30 times, at least 40 times, at least 50 times, at least 75 times, at least 100 times, at least 125 times, at least 150 times, at least 175 times, at least 200 times or 300 times or great efficiency than said binding agent binds CD32A. For example, such antibodies may be, may be derived from (e.g., humanized or chimerized versions of), or compete for binding with a monoclonal antibody produced by clones 1D5, 2E1, 2H9, 2D11, and 1F2 having ATCC Accession numbers, PTA-5958, PTA-5961, PTA-5962, PTA-5960, and PTA-5959, respectively. In certain embodiments, the anti-CD32B antibody is, is derived from, or competed for binding with an antibody produced by clone 2B6 or 3H7 which bind Fc□RIIB, having ATCC accession numbers PTA-4591 or PTA-4592, respectively. In other embodiments the invention encompasses a humanized version of antibody 2B6, encoded by plasmid pMGx0675, having ATCC accession number PTA-7609. In particular embodiments, said CD32B binding agent is an anti-CD32B antibody that has an Fc domain comprising at least one amino-acid modification relative to a wild type Fc domain, or an analog, derivative, or fragment thereof. In particular embodiments, CD32B antibody that has an Fc domain comprising at least one amino-acid modification relative to a wild type Fc domain, or an analog, derivative, or fragment thereof, exhibits altered ADCC activity relative to the wild-type antibody, analog, derivative, or fragment thereof. In other embodiments, the antibody lacks an Fc domain (e.g., is a Fab fragment, F(ab′)₂ or single chain antibody.

The invention comprises methods wherein the expression of CD32B and/or CD138 are characterized in a population of plasma cells from a subject. In certain embodiments, the expression of CD138 and/or CD32B is detected in said population. In other embodiments, the levels of expression are determined in said population. In certain embodiments, CD138 and/or CD32B are detected and/or characterized by any method known in the art, e.g., methods comprising the use of an anti-CD138 and/or anti-CD32B antibodies, respectively, or antigen binding fragments thereof. In certain embodiments, the antibodies or antigen binding fragments thereof further comprises a detectable marker. In related embodiments, the detectable marker may be a chemiluminescent, enzymatic, fluorescent, or radioactive label, and said label detected by any method described herein or known in the art, e.g., fluorescence activated cell sorting (FACS) or flow cytometric analysis (FCM). In certain embodiments, a specific cell population, preferably a CD138 positive cell population, may be isolated from said biological sample based on expression of a specific surface marker, e.g., CD138. In other embodiments, the expression of CD138 and/or CD32B is determined by use of an anti-CD138 antibody and/or an anti-CD32B antibody, respectively, or fragment thereof that comprises a detectable marker and determining the level of said marker by any method known in the art. For example, one such detectable marker is a fluorescent label that may be conjugated to an antibody. Cells stained with the marker may be measured for level of said marker or sorted from non-stained cells by any method known in the art, e.g., fluorescence activated cell sorting (FACS) or flow cytometric analysis (FCM).

The invention provides methods comprising the use of a biological sample containing plasma cells from a subject. In certain embodiments, the biological sample is a blood sample. In other embodiments, the biological sample is a sample of bone marrow aspirate. In yet other embodiments, the biological sample has been heparinized. In still other embodiments, the biological sample is or has been frozen.

The present invention provides methods of treating, preventing, preventing the progression of and/or ameliorating a plasma cell disorder characterized by the expression of CD32B, or one or more symptoms thereof, particularly systemic light-chain amyloidosis (AL), multiple myeloma (MM), and plasma cell leukemia (PCL), by administering to a subject a therapeutically or prophylactic ally acceptable amount of a CD32B binding agent. In particular, the methods of the invention provide for administration of antibodies, analogs, derivatives, or fragments thereof that immunospecifically bind CD32B. For example, such antibodies may be, may be derived from (e.g., humanized or chimerized versions of), or compete with a monoclonal antibody produced by clones 1D5, 2E1, 2H9, 2D11, and 1F2 having ATCC Accession numbers, PTA-5958, PTA-5961, PTA-5962, PTA-5960, and PTA-5959, respectively. In certain embodiments, the anti-CD32B antibody is, is derived from, or competed for binding with an antibody produced by clone 2B6 or 3H7 which bind Fc□RIIB, having ATCC accession numbers PTA-4591 or PTA-4592, respectively. In other embodiments the invention encompasses a humanized version of antibody 2B6, encoded by plasmid pMGx0675, having ATCC accession number PTA-7609. In particular embodiments, the methods of the invention encompass the prior, concomitant, or subsequent administration of an additional cancer therapy. In specific embodiments, the additional cancer therapy is antibody therapy, cytokine therapy, chemotherapy, hematopoietic stem cell transplantation, B-cell mediated therapy, biological therapy, immunotherapy, radiation therapy, hormonal therapy or surgery.

The invention particularly provides methods of treating, preventing or ameliorating a plasma cell disorder, or one or more symptoms thereof, in particular, systemic light-chain amyloidosis (AL), multiple myeloma (MM) or plasma cell leukemia (PCL). Particularly, the methods of the invention are advantageous in subjects with early stage disease to slow or prevent the progression of the disease, to slow or prevent the damage to tissues or organs from the accumulation of amyloid protein, and/or to reduce the need for other therapy. In specific embodiments, the methods of the invention prevent or slow the progression of said plasma cell disorder to a more aggressive disease state. For example, the treatment of a subject according to the methods of the invention may prevent or delay the progression of multiple myeloma (MM) from an asymptomatic state, e.g., monoclonal gammopathies of undetermined significance (MGUS), smoldering multiple myeloma (SMM), indolent multiple myeloma or early stage MM (stage I), to a later stage MM (stage II or stage III) in a subject by 2 months, 4 months, 6 months, 8, months, 10 months, 12 months, 15 months, 18 months, 21 months, 24 months, 2.5 years, 3 years, 4 years, 5 years, 6 years, 8 years, or 10 years or longer relative to a subject with similar clinical parameters who did not receive treatment. In a specific embodiment, the treatment of a subject according to the methods of the invention may prevent or delay the progression of gammopathies of undetermined significance (MGUS) to multiple myeloma (MM) in a subject by 2 months, 4 months, 6 months, 8, months, 10 months, 12 months, 15 months, 18 months, 21 months, 24 months, 2.5 years, 3 years, 4 years, 5 years, 6 years, 8 years, or 10 years or longer relative to a subject with similar clinical parameters who did not receive treatment. In another embodiment, the treatment of a subject according to the methods of the invention may prevent or delay the progression of multiple myeloma (MM) to plasma cell leukemia (PCL) in a subject by 2 months, 4 months, 6 months, 8, months, 10 months, 12 months, 15 months, 18 months, 21 months, 24 months, 2.5 years, 3 years, 4 years, 5 years, 6 years, 8 years, or 10 years or longer relative to a subject with similar clinical parameters who did not receive treatment.

Symptoms of plasma cell disorders, e.g. AL, MM or PCL, include, but are not limited to presence of amyloid protein (M protein) in the serum, decrease in hemoglobin value, increase in serum calcium concentration, presence of osteolytic lesions, and myeloma accumulation in the marrow. In a specific embodiment, a CD32B binding agent, e.g., an antibody, analog, derivative, or fragment thereof, used in the methods of the invention, when administered alone or in combination with a cytotoxic therapeutic antibody, inhibits or reduces the growth or metastasis of malignant cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth or metastasis in absence of said binding agent used in the methods of the invention. In another embodiment, a CD32B binding agent, e.g., an antibody, analog, derivative, or fragment thereof, used in the methods of the invention, when administered alone or in combination with a cytotoxic therapeutic antibody, inhibits or reduces the proliferation of malignant plasma cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the proliferation of said cells in the absence of said binding agent used in the methods of the invention. In other embodiments, a CD32B binding agent, e.g., an antibody, analog, derivative, or fragment thereof, used in the methods of the invention, when administered alone or in combination with a cytotoxic therapeutic antibody, inhibits or reduces the concentration of serum M protein is a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the concentration of serum M protein in said subject absence of said binding agent used in the methods of the invention. In still other embodiment, a CD32B binding agent, e.g., an antibody, analog, derivative, or fragment thereof, used in the methods of the invention, when administered alone or in combination with a cytotoxic therapeutic antibody, increases the hemoglobin value of a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the hemoglobin value of said subject in absence of said binding agent used in the methods of the invention. In other embodiments, a CD32B binding agent, e.g., an antibody, analog, derivative, or fragment thereof, used in the methods of the invention, when administered alone or in combination with a cytotoxic therapeutic antibody, reduces the total number and/or severity of one or more lytic bone lesions in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to that of said subject in the absence of said binding agent used in the methods of the invention. In other embodiments, a CD32B binding agent, e.g., an antibody, analog, derivative, or fragment thereof, used in the methods of the invention, when administered alone or in combination with a cytotoxic therapeutic antibody, reduces the concentration of serum calcium in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the concentration of serum calcium in said subject in the absence of said binding agent used in the methods of the invention.

The methods of the invention may further be used to treat, prevent, manage, or ameliorate an autoimmune disease, or one of more symptoms thereof. The present invention provides methods of preventing, treating, or managing one or more symptoms associated with an autoimmune disorder in a subject, comprising administering to said subject a therapeutically effective amount of the CD32B binding agent of the invention, e.g., a humanized anti-CD32B antibody, analog, derivative, or fragment thereof. In specific embodiments, the autoimmune disease is rheumatoid arthritis, multiple sclerosis, lupus or type-1 diabetes.

In certain embodiments, the methods of the invention provide for identification or treatment of a subject that has a predisposition of developing a plasma cell disorder, but has not yet been diagnosed with said disorder (according to criteria accepted in the art) and/or has not yet experienced the clinical symptoms of said disorder. In specific examples in accordance with this embodiment, administration of a binding agent of the invention, preferably an anti CD32B antibody, analog, derivative or fragment thereof, prevents onset and/or development of the disorder or prevents onset of symptoms of the disorder by 2 months, 4 months, 6 months, 8, months, 10 months, 12 months, 15 months, 18 months, 21 months, or 24 months relative to a subject with similar clinical parameters who did not receive treatment. In certain embodiments, administration of a binding agent of the invention, preferably an anti CD32B antibody, analog, derivative or fragment thereof, prevents onset and/or development of the disorder, prevents onset of symptoms of the disorder, and/or delays the positive diagnosis of said disorder by 2 months, 4 months, 6 months, 8, months, 10 months, 12 months, 15 months, 18 months, 21 months, or 24 months relative to a subject with similar clinical parameters who did not receive treatment. In other embodiments, administration of a binding agent of the invention, preferably an anti CD32B antibody, analog, derivative or fragment thereof, prolongs survival of said subject after diagnosis with said disorder by 2 months, 4 months, 6 months, 8, months, 10 months, 12 months, 15 months, 18 months, 21 months, or 24 months relative to a subject with similar clinical parameters who did not receive treatment. In a specific example in accordance with this embodiment, the subject to be treated with an antibody of the invention does not meet the criteria for diagnosis of multiple myeloma (see, e.g., Mihou et al., 2006, Haematologica 91:1149-1150, hereby incorporated by reference in its entirety). In a specific example, the subject with the predisposition for developing multiple myeloma is a subject diagnosed with monoclonal gammopathies of undetermined significance (MGUS). In specific embodiments, the methods of the invention provide In specific embodiments, said administration is intravenous, subcutaneous, intramuscular, oral or intranasal.

The CD32B binding agents of the invention may exhibit cytotoxic activity, or exhibit no cytotoxic activity. For example, where the CD32B binding agent is an anti-CD32B antibody, analog, derivative, or fragment thereof, said agent will exhibit ADCC activity and elicit cell-killing activity toward cells expressing CD32B. In certain embodiments, the CD32B binding agent may be conjugated to cytotoxic agents. In certain embodiments, the cytotoxic agent is paclitaxel, cytochalasin B, gramicidin D, ethidium bromide, emetine, mitomycin, carmustine, etoposide, tenoposide, vincristine, vinblastine, colchicine, doxorubicin, daunorubicin, dihydroxy anthracin dione, mitoxantrone, mithramycin, melphalan, actinomycin D, 1-dehydrotestosterone, glucocorticoids, procaine, tetracaine, lidocaine, propranolol, puromycin, epirubicin, or cyclophosphamide. The CD32B binding agents of the invention, preferably, anti-CD32B antibodies, analogs, derivatives, or fragments thereof, may be further conjugated to any therapeutic known in the art for treatment, prevention, management, or amelioration of a plasma cell disorder, or a symptom thereof. IN specific examples, the therapeutic agent is a heterologous polypeptide or an antibody that immunospecifically binds to a cell surface receptor other than CD32B.

In a particular embodiment, the CD32B binding agents of the invention, preferably, anti-CD32B antibodies, analogs, derivatives, or fragments thereof, agonize at least one activity of FcγRIIB. In one embodiment of the invention, said activity is inhibition of B cell receptor-mediated signaling. In another embodiment, CD32B binding agents of the invention, preferably, anti-CD32B antibodies, analogs, derivatives, or fragments thereof, inhibit activation of B cells, B cell proliferation, antibody production, intracellular calcium influx of B cells, cell cycle progression, or activity of one or more downstream signaling molecules in the FcγRIIB signal transduction pathway. In yet another embodiment, the CD32B binding agents of the invention, preferably, anti-CD32B antibodies, analogs, derivatives, or fragments thereof, enhance phosphorylation of FcγRIIB or SHIP recruitment. In a further embodiment of the invention, the CD32B binding agents of the invention, preferably, anti-CD32B antibodies, analogs, derivatives, or fragments thereof, inhibit MAP kinase activity or Akt recruitment in the B cell receptor-mediated signaling pathway. In another embodiment, the humanized agonistic antibodies of the invention agonize FcγRIIB-mediated inhibition of FcεRI signaling. In a particular embodiment, said CD32B binding agents of the invention, preferably, anti-CD32B antibodies, analogs, derivatives, or fragments thereof, inhibit FcεRI-induced mast cell activation, calcium mobilization, degranulation, cytokine production, or serotonin release. In another embodiment, the CD32B binding agents of the invention, preferably, anti-CD32B antibodies, analogs, derivatives, or fragments thereof, stimulate phosphorylation of FcγRIIB, stimulate recruitment of SHIP, stimulate SHIP phosphorylation and its association with Shc, or inhibit activation of MAP kinase family members (e.g., Erk1, Erk2, JNK, p38, etc.). In yet another embodiment, the CD32B binding agents of the invention, preferably, anti-CD32B antibodies, analogs, derivatives, or fragments thereof, enhance tyrosine phosphorylation of p62dok and its association with SHIP and rasGAP. In another embodiment, the CD32B binding agents of the invention, preferably, anti-CD32B antibodies, analogs, derivatives, or fragments thereof, inhibit FcγR-mediated phagocytosis in monocytes or macrophages.

In another embodiment, the CD32B binding agents of the invention, preferably, anti-CD32B antibodies, analogs, derivatives, or fragments thereof, antagonize at least one activity of FcγRIIB. In one embodiment, said activity is activation of B cell receptor-mediated signaling. In a particular embodiment, the CD32B binding agents of the invention, preferably, anti-CD32B antibodies, analogs, derivatives, or fragments thereof, enhance B cell activity, B cell proliferation, antibody production, intracellular calcium influx, or activity of one or more downstream signaling molecules in the FcγRIIB signal transduction pathway. In yet another particular embodiment, the CD32B binding agents of the invention, preferably, anti-CD32B antibodies, analogs, derivatives, or fragments thereof, decrease phosphorylation of FcγRIIB or SHIP recruitment. In a further embodiment of the invention, the CD32B binding agents of the invention, preferably, anti-CD32B antibodies, analogs, derivatives, or fragments thereof, enhance MAP kinase activity or Akt recruitment in the B cell receptor mediated signaling pathway. In another embodiment, the CD32B binding agents of the invention, preferably, anti-CD32B antibodies, analogs, derivatives, or fragments thereof, antagonize FcγRIIB-mediated inhibition of FcεRI signaling. In a particular embodiment, the CD32B binding agents of the invention, preferably, anti-CD32B antibodies, analogs, derivatives, or fragments thereof, enhance FcεRI-induced mast cell activation, calcium mobilization, degranulation, cytokine production, or serotonin release. In another embodiment, the CD32B binding agents of the invention, preferably, anti-CD32B antibodies, analogs, derivatives, or fragments thereof, inhibit phosphorylation of FcγRIIB, inhibit recruitment of SHIP, inhibit SHIP phosphorylation and its association with Shc, enhance activation of MAP kinase family members (e.g., Erk1, Erk2, JNK, p38, etc.). In yet another embodiment, the CD32B binding agents of the invention, preferably, anti-CD32B antibodies, analogs, derivatives, or fragments thereof, tyrosine phosphorylation of p62dok and its association with SHIP and rasGAP. In another embodiment, the CD32B binding agents of the invention, preferably, anti-CD32B antibodies, analogs, derivatives, or fragments thereof, enhance FcγR-mediated phagocytosis in monocytes or macrophages. In another embodiment, the CD32B binding agents of the invention, preferably, anti-CD32B antibodies, analogs, derivatives, or fragments thereof, prevent phagocytosis, clearance of opsonized particles by splenic macrophages.

As evidenced by a survey of the art, there remains a need for methods diagnosing, treating, managing, or preventing plasma cell disorders such as plasma cell malignancies.

### 3.1 DEFINITIONS

As used herein, the terms “plasma cell malignancies” and “plasma cell malignancy” refer to any plasma cell lymphoproliferative disorder. Plasma cell malignancies include tumors of plasma cell origin. Plasma cell disorders include, but are not limited to monoclonal gammopathies of undetermined significance (MGUS), multiple myeloma (MM), macroglobulinemia, heavy chain diseases, systemic light-chain amyloidosis (AL), and plasma cell leukemia (PCL).

As used herein, the term “specifically binds to FcγRIIB” and analogous terms refer to antibodies or fragments thereof (or any other FcγRIIB binding molecules) that specifically bind to FcγRIIB or a fragment thereof and do not specifically bind to other Fc receptors, in particular to FcγRIIA. Further it is understood to one skilled in the art, that an antibody that specifically binds to FcγRIIB, may bind through the variable domain. If the antibody that specifically binds to FcγRIIB binds through its variable domain, it is understood to one skilled in the art that it is not aggregated, i.e., is monomeric. An antibody that specifically binds to FcγRIIB may bind to other peptides or polypeptides with lower affinity as determined by, e.g., immunoassays, BIAcore, or other assays known in the art. Preferably, antibodies or fragments that specifically bind to FcγRIIB or a fragment thereof do not cross-react with other antigens. Antibodies or fragments that specifically bind to FcγRIIB can be identified, for example, by immunoassays, BIAcore, or other techniques known to those of skill in the art. An antibody or a fragment thereof binds specifically to a FcγRIIB when it binds to FcγRIIB with higher affinity than to any cross-reactive antigen as determined using experimental techniques, such as western blots, radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

As used herein, the term “native FcγRIIB” refers to FcγRIIB which is endogenously expressed and present on the surface of a cell. In some embodiments, “native FcγRIIB” encompasses a protein that is recombinantly expressed in a mammalian cell. Preferably, the native FcγRIIB is not expressed in a bacterial cell, i.e., E. coli. Most preferably the native FcγRIIB is not denatured for example, with SDS or other chaotropic agent, i.e., it is in its biologically active conformation.

As used herein, the term “native FcγRIIA” refers to FcγRIIA which is endogenously expressed and present on the surface of a cell. In some embodiments, “native FcγRIIA” encompasses a protein that is recombinantly expressed in a mammalian cell. Preferably, the native FcγRIIA is not expressed in a bacterial cell, i.e., E. coli. Most preferably the native FcγRIIA is not denatured, i.e., it is in its biologically active conformation.

As used herein, the term “analog” in the context of proteinaceous agents (e.g., proteins, polypeptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a polypeptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions in the alignment of both proteins×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous agent refers to a second organic or inorganic molecule which possess a similar or identical function as a first organic or inorganic molecule and is structurally similar to the first organic or inorganic molecule.

As used herein, the terms “antagonist” and “antagonists” refer to any protein, polypeptide, peptide, antibody, antibody fragment, large molecule, or small molecule (less than 10 kD) that blocks, inhibits, reduces or neutralizes a function, activity and/or expression of another molecule, such as that of CD32B. In various embodiments, an antagonist reduces a function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the terms “agonist” and “agonists” refer to any protein, polypeptide, peptide, antibody, antibody fragment, large molecule, or small molecule (less than 10 kD) that elicits, induces, activates, or increases a function, activity and/or expression of another molecule, such as that of CD32B. In various embodiments, an agonist increases a function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the terms “aggressivity” and “biologic aggresivity” in reference to a disease or disorder involving malignant or neoplastic cells refer to an evaluation of the phenotype of the malignant cells relative to the expected phenotype of a non-malignant, e.g., normal, cell of the same lineage. Malignant cells may be classified or staged by a number of phenotypic characteristics relative to normal cells including, but not limited to insensitivity to apoptotic signals, presence/absence of autocrine regulatory pathways, insensitivity to anti-growth signals, capability for tissue invasion/metastasis, increased replicative potential, decreased doubling time, expression/lack of differentiation markers, and increased angiogenic potential. The greater the total number of such characteristics and/or the greater the divergence of said characteristic from the expected norm, i.e., said characteristic in a normal cell of the same lineage, the more “aggressive” the cell population will be considered.

As used herein, the terms “antibody” and “antibodies” refer to monoclonal antibodies, multispecific antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies (including, e.g., anti-Id and anti-anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

As used herein, the term “derivative” refers to an antibody that comprises an amino acid sequence which has been altered by the introduction of amino acid residue substitutions, deletions or additions. The term “derivative” as used herein also refers to an antibody which has been modified, i.e, by the covalent attachment of any type of molecule to the antibody. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative antibody may be produced by chemical modifications using techniques known to those of skill in the art, including, but not limited to specific chemical cleavage, acetylation, formylation, metabolic synthesis of tunicamycin, etc. Further, a derivative antibody possesses a similar or identical function as the antibody from which it was derived.

The term “derivative” as used herein in conjunction with FcγRIIB refers to an antibody that immunospecifically binds to a FcγRIIB polypeptide, or an antibody fragment that immunospecifically binds to a FcγRIIB polypeptide, that has been altered by the introduction of amino acid residue substitutions, deletions or additions (i.e., mutations). In some embodiments, an antibody derivative or fragment thereof comprises amino acid residue substitutions, deletions or additions in one or more CDRs. The antibody derivative may have substantially the same binding, better binding, or worse binding when compared to a non-derivative antibody. In specific embodiments, one, two, three, four, or five amino acid residues of the CDR have been substituted, deleted or added (i.e., mutated). The term “derivative” as used herein in conjunction with FcγRIIB also refers to an antibody that immunospecifically binds to a FcγRIIB polypeptide, or an antibody fragment that immunospecifically binds to a FcγRIIB polypeptide which has been modified, i.e., by the covalent attachment of any type of molecule to the polypeptide. For example, but not by way of limitation, an antibody, or antibody fragment may be modified, e.g., by glycosylation, acetylation, pegylation, phosphorylation, amidation, derivatization by known protecting/blocking groups, proteolytic cleavage, linkage to a cellular ligand or other protein, etc. A derivative antibody, or antibody fragment may be modified by chemical modifications using techniques known to those of skill in the art, including, but not limited to, specific chemical cleavage, acetylation, formulation, metabolic synthesis of tunicamycin, etc. Further, a derivative of an antibody, or antibody fragment may contain one or more non-classical amino acids. In one embodiment, an antibody derivative possesses a similar or identical function as the parent antibody. In another embodiment, a derivative of an antibody, or antibody fragment has an altered activity when compared to an unaltered antibody. For example, a derivative antibody or fragment thereof can bind to its epitope more tightly or be more resistant to proteolysis.

As used herein, the terms “disorder” and “disease” are used interchangeably to refer to a condition in a subject, in particular a condition associated with the aberrant growth or activity of a plasma cell or cell of plasma cell lineage. In particular, the term “plasma cell disease” is used interchangeably with the term “plasma cell disorder” to refer to a condition in a subject characterized by cellular, tissue and/or organ injury caused by infiltration and growth of plasma cells or the accumulation and/or deposition of amyloid protein. With reference to a disease associated with autoimmunity, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs.

As used herein, the term “cancer” refers to a neoplasm or tumor resulting from abnormal uncontrolled growth of cells. As used herein, cancer includes, but is not limited to, multiple myeloma and plasma cell leukemia. The term “cancer” also refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen.

As used herein, the term “immunomodulatory agent” and variations thereof including, but not limited to, immunomodulatory agents, refer to an agent that modulates a host's immune system. In certain embodiments, an immunomodulatory agent is an immunosuppressant agent. In certain other embodiments, an immunomodulatory agent is an immunostimulatory agent. Immunomodatory agents include, but are not limited to, small molecules, peptides, polypeptides, fusion proteins, antibodies, inorganic molecules, mimetic agents, and organic molecules.

As used herein, the term “epitope” refers to region on an antigen molecule to which an antibody binds specifically.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide. In a specific embodiment, a fragment of a polypeptide retains at least one function of the polypeptide. Preferably, antibody fragments are epitope binding fragments.

As used herein, the term “humanized antibody” refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody, because, e.g., the entire variable region of a chimeric antibody is non-human. One says that the donor antibody has been “humanized”, by the process of “humanization”, because the resultant humanized antibody is expected to bind to the same antigen as the donor antibody that provides the CDR's. For the most part, humanized antibodies are human immunoglobulins (recipient antibody) in which hypervariable region residues of the recipient are replaced by hypervariable region residues from a non-human species (donor antibody) such as mouse, rat, rabbit or a non-human primate having the desired specificity, affinity, and capacity. In some instances, Framework Region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin that immunospecifically binds to a CD32B polypeptide, that has been altered by the introduction of amino acid residue substitutions, deletions or additions (i.e., mutations). In some embodiments, a humanized antibody is a derivative. Such a humanized antibody comprises amino acid residue substitutions, deletions or additions in one or more non-human CDRs. The humanized antibody derivative may have substantially the same binding, better binding, or worse binding when compared to a non-derivative humanized antibody. In specific embodiments, one, two, three, four, or five amino acid residues of the CDR have been substituted, deleted or added (i.e., mutated). For further details in humanizing antibodies, see European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-25; Caldas et al., 2000, Protein Eng. 13:353-60; Morea et al., 2000, Methods 20:267-79; Baca et al., 1997, J. Biol. Chem. 272:10678-84; Roguska et al., 1996, Protein Eng. 9:895-904; Couto et al., 1995, Cancer Res. 55 (23 Supp):5973s-5977s; Couto et al., 1995, Cancer Res. 55:1717-22; Sandhu, 1994, Gene 150:409-10; Pedersen et al., 1994, J. Mol. Biol. 235:959-73; Jones et al., 1986, Nature 321:522-525; Reichmann et al., 1988, Nature 332:323-329; and Presta, 1992, Curr. Op. Struct. Biol. 2:593-596.

As used herein, the terms “hyperproliferative cell disorder,” “hyperproliferative cell disease,” “hyperproliferative disorder,” and “hyperproliferative disease” and analogous terms refer to a disorder in which cellular hyperproliferation or any form of excessive cell accumulation causes or contributes to the pathological state or symptoms of the disorder. In an embodiment, the hyperproliferative cell disorder is characterized by hyperproliferating plasma cells. In certain embodiments, the hyperproliferative cell disorder is not malignant or neoplastic. In certain embodiments, the hyperproliferative cell disorder is malignant or neoplastic. In certain embodiments, the hyperproliferative disorder is multiple myeloma.

As used herein, the term “hypervariable region” refers to the amino acid residues of an antibody which are responsible for antigen binding. The hypervariable region comprises amino acid residues from a “Complementarity Determining Region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). CDR residues for Eph099B-208.261 and Eph099B-233.152 are listed in Table 1. “Framework Region” or “FR” residues are those variable domain residues other than the hypervariable region residues as herein defined.

As used herein, the terms “single-chain Fv” or “scFv” refer to antibody fragments comprise the VH and VL domains of antibody, wherein these domains are present in a single polypeptide chain. Generally, the Fv polypeptide further comprises a polypeptide linker between the VH and VL domains which enables the scFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994). In specific embodiments, scFvs include bi-specific scFvs and humanized scFvs.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the term “neoplastic” refers to a disease involving cells that have the potential to metastasize to distal sites. Neoplastic cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. Thus, “non-neoplastic” means that the condition, disease, or disorder does not involve cancer cells.

As used herein, the terms “subject” and “subject” are used interchangeably. As used herein, a subject is preferably a mammal such as a non-primate (e.g., cows, pigs, horses, cats, dogs, rats etc.) and a primate (e.g., monkey and human), most preferably a human.

As used herein, the terms “treat,” “treating” and “treatment” refer to the eradication, reduction or amelioration of symptoms of a disease or disorder related to the loss of regulation in the Fc receptor signaling pathway or to enhance the therapeutic efficacy of another therapy, e.g., a therapeutic antibody, vaccine therapy or prophylaxis. In some embodiments, treatment refers to the eradication, removal, modification, or control of primary, regional, or metastatic cancer tissue that results from the administration of one or more therapeutic agents. In certain embodiments, such terms refer to the minimizing or delaying the spread of cancer resulting from the administration of one or more therapeutic agents to a subject with such a disease. In other embodiments, such terms refer to elimination of disease causing cells.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by subjects are numerous and known in the art, see, e.g., the Physicians' Desk Reference (56^{th} ed., 2002), which is incorporated herein by reference in its entirety.

As used herein, a “therapeutically effective amount” refers to that amount of the therapeutic agent sufficient to treat or manage a disease or disorder associated with or characterized by CD32B expression and/or any disease related to the loss of regulation in the Fc receptor signaling pathway or to enhance the therapeutic efficacy of another therapy, e.g., therapeutic antibody, vaccine therapy or prophylaxis, etc. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease. Further, a therapeutically effective amount with respect to a therapeutic agent of the invention means that amount of therapeutic agent alone, or in combination with other therapies, that provides a therapeutic benefit in the treatment or management of a disease, e.g., sufficient to enhance the therapeutic efficacy of a therapeutic antibody sufficient to treat or manage a disease. Used in connection with an amount of CD32B antibody, the term can encompass an amount that improves overall therapy, reduces or avoids unwanted effects, or enhances the therapeutic efficacy of or synergies with another therapeutic agent.

As used herein, the terms “prophylactic agent” and “prophylactic agents” refer to any agent(s) which can be used in the prevention of a disorder, or prevention of recurrence or spread of a disorder. A prophylactically effective amount may refer to the amount of prophylactic agent sufficient to prevent the recurrence or spread of hyperproliferative disease, particularly cancer, or the occurrence of such in a subject, including but not limited to those predisposed to hyperproliferative disease, for example those genetically predisposed to cancer or previously exposed to carcinogens. A prophylactically effective amount may also refer to the amount of the prophylactic agent that provides a prophylactic benefit in the prevention of disease. Further, a prophylactically effective amount with respect to a prophylactic agent of the invention means that amount of prophylactic agent alone, or in combination with other agents, that provides a prophylactic benefit in the prevention of disease. Used in connection with an amount of an FcγRIIB antibody of the invention, the term can encompass an amount that improves overall prophylaxis or enhances the prophylactic efficacy of or synergies with another prophylactic agent, such as but not limited to a therapeutic antibody. In certain embodiments, the term “prophylactic agent” refers to an agonistic FcγRIIB-specific antibody. In other embodiments, the term “prophylactic agent” refers to an antagonistic FcγRIIB-specific antibody. In certain other embodiments, the term “prophylactic agent” refers to cancer chemotherapeutics, radiation therapy, hormonal therapy, biological therapy (e.g., immunotherapy), and/or FcγRIIB antibodies of the invention. In other embodiments, more than one prophylactic agent may be administered in combination.

As used herein, the terms “manage,” “managing” and “management” refer to the beneficial effects that a subject derives from administration of a prophylactic or therapeutic agent, which does not result in a cure of the disease. In certain embodiments, a subject is administered one or more prophylactic or therapeutic agents to “manage” a disease so as to prevent the progression or worsening of the disease.

As used herein, the terms “prevent”, “preventing” and “prevention” refer to the prevention of the occurrence and/or recurrence or onset of one or more symptoms of a disorder in a subject resulting from the administration of a prophylactic or therapeutic agent.

As used herein, the term “in combination” refers to the use of more than one prophylactic and/or therapeutic agents. The use of the term “in combination” does not restrict the order in which prophylactic and/or therapeutic agents are administered to a subject with a disorder, e.g., hyperproliferative cell disorder, especially cancer. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to a disorder. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents.

### 4. BRIEF DESCRIPTION OF THE DRAWINGS

FIGS. 1 A-D. Representative FACS plots of plasma cells from subjects with (A) systemic light-chain amyloidosis (AL); (B) multiple myeloma (MM) with favorable cytogenetics; (C) multiple myeloma (MM) with unfavorable cytogenetics; and (D) plasma cell leukemia (PCL). For all plots, plasma cells were identified by expression of CD138. Intensity of CD138 is indicated on the x-axis and intensity for CD32B staining is indicated on the y-axis. The percentage of CD138 positive cells that express CD32B is indicated in each plot.

### 5. DETAILED DESCRIPTION OF THE INVENTION

The present invention encompasses methods for the diagnosis, treatment, management or prevention of plasma cell disorders, and, in particular, systemic light-chain amyloidosis (AL), multiple myeloma (MM), and plasma cell leukemia (PCL). The methods described herein provide better prophylactic and therapeutic profiles than current therapies for a plasma cell disorder associated with CD32B expression, or one or more symptoms thereof. In accordance with the methods of the present invention agents that bind and modulate the expression and/or function of CD32B may be used for the prevention, treatment, management, or amelioration of a plasma cell disorder, or one or more symptoms thereof. In particular, the invention provides prophylactic and therapeutic protocols for the prevention, treatment, management, or amelioration of a plasma cell disorder, or one or more symptoms thereof, comprising the administration of CD32B binding agents to a subject in need thereof. In certain embodiments the CD32B binging agent is an anti-CD32B antibody, analog, derivative, or fragment thereof. In particular embodiments, methods of the inventions are used to treat, manage, prevent, or ameliorate systemic light-chain amyloidosis (AL), or one or more symptoms thereof. In another embodiment, the methods of the invention are used to diagnose, treat, manage, prevent, or ameliorate multiple myeloma (MM), or one or more symptoms thereof. In another embodiment, the methods of the invention are used to diagnose, treat, manage, prevent, or ameliorate plasma cell leukemia (PCL), or one or more symptoms thereof.

The present invention also encompasses methods for the diagnosis, screening or monitoring the progression of plasma cell disorders comprising the use of CD32B binding agents, in particular, anti-CD32B antibodies or an antigen binging fragment thereof. In certain embodiments, the use of CD32B binding agents, preferably, anti-CD32B antibodies or an antigen binging fragment thereof, permits the characterization and detection of plasma cells with aberrant or altered expression of CD32B. In certain embodiments, the expression of CD32B of a plasma cell population may be compared with a known standard, e.g., the expression of CD32B on a known non-malignant plasma cell, such that the degree of altered expression is ascertained. Because the degree of altered expression correlates with the progression of disease and/or aggressivity of the plasma cell disorder, the instant invention allows for treatment options to be better evaluated than current methods of staging plasma cell disease.

The CD32B binding agents of the invention may also be used to target CD32B expressing cells, preferably plasma cells, that cause or are associated with other diseases associated with aberrant, altered, or undesired antibody expression, e.g., autoimmune disorders. In particular, methods of the presents invention encompass the use of CD32B binding agents that have cytotoxic activity to target antibody producing plasma cells and, in particular, those plasma cell clones that express auto-reactive antibodies. Accordingly, the present invention encompasses methods for the treatment, management or prevention of autoimmune disorders. The methods described herein provide improved prophylactic and therapeutic profiles over current therapies, e.g., general immunosuppressive therapies, for treatment of autoimmune disorders, or one or more symptoms thereof. In accordance with the methods of the present invention, agents that bind and modulate the expression and/or function of CD32B may be used for the prevention, treatment, management, or amelioration of an autoimmune disorder, or one or more symptoms thereof. In particular, the invention provides prophylactic and therapeutic protocols for the prevention, treatment, management, or amelioration of an autoimmune disorder, or one or more symptoms thereof, comprising the administration of CD32B binding agents to a subject in need thereof. In particular embodiments, the CD32B binding agent binds to CD32B expressed on the surface of plasma cells that express and/or produce auto-reactive antibodies. In certain embodiments the CD32B binging agent is an anti-CD32B antibody, analog, derivative, or fragment thereof. In certain embodiments, the anti-CD32B antibody, analog, derivative, or fragment thereof comprises at least one amino acid modification relative to a wild-type anti-CD32B antibody, analog, derivative, or fragment thereof, such that said antibody exhibits altered, preferably enhanced, ADCC activity relative to the wild-type antibody as assayed in vitro by any method known in the art. In specific embodiments, the invention encompasses prophylactic and therapeutic methods for the treatment, prevention, management or amelioration of an autoimmune disorder, or one or more symptoms thereof, comprising the administration of an anti-CD32B antibody, analog, derivative or fragment thereof to a subject in need thereof, wherein the anti-CD32B antibody, analog, derivative, or fragment thereof exhibits ADCC activity as assayed by any in vitro method described herein or known in the art.

The present invention also provides pharmaceutical compositions and kits comprising CD32B binding agents for use in the diagnosis, prevention, treatment, management, or amelioration of a plasma cell disorder or autoimmune disorder, or one or more symptoms thereof. In certain embodiments, the CD32B binding agent is an anti-CD32B antibody, analog, derivative, or fragment. In particular, the present invention provides pharmaceutical compositions and kits comprising a an anti-CD32B antibody or an antigen-binding fragment thereof.

5.1 Plasma Cell Disorders

The methods of the invention are useful for diagnosing, treating, managing or preventing any plasma cell disorder characterized by the aberrant or altered expression of CD32B relative to a non-malignant cell or non-diseased cell, particularly amyloidosis, multiple myeloma and plasma cell leukemia. In other embodiments, the plasma cell disorder is characterized by plasma cells with a normal pattern or level of CD32B expression. Plasma cell disorders include monoclonal gammopathies of undetermined significance (MGUS), multiple myeloma (MM), smoldering multiple myeloma (SMM), indolent multiple myeloma (IMM), macroglobulinemia, heavy chain diseases, systemic light-chain amyloidosis (AL), and plasma cell leukemia (PCL). Although variable, plasma cell disorders can present as anemia, hypercalcemia, amyloid deposits, lytic bone lesions, and presence of serum M protein.

As demonstrated by the instant invention, CD32B is found to be expressed at a high level on plasma cells isolated from AL subjects and in the majority of plasma cells isolated from subjects with MM showing normal karyotype and FISH; at a moderate level on plasma cells isolated from subjects with MM showing poor karyotypic prognostic indicators; and at a very low level to undetectable level on a minority of the cells isolated from subjects with PCL. Thus CD32B expression may be used to target plasma cells associated with plasma cell disorders, especially at early stages of the disease. Because CD32B expression correlates with disease state, e.g., aggressivity, methods of the invention are, in particular, directed to the targeting of early stage disease and preventing progression to a more aggressive disease state. In certain embodiments, CD32B binding agents are used in combination with cytotoxins, e.g., conjugated to a cytotoxin, to treat or prevent plasma cell disorders characterized by the expression of CD32B. Additionally, because CD32B is a cell receptor associated with inhibitory signaling, CD32B binding agents may also be used to treat, prevent, or manage a plasma cell disorder associated with CD32B expression by modulating activity and/or expression of CD32B. Although not intending to be bound by any mechanism of action, the CD32B binding agents of the invention inhibit or prevent plasma cell disorders by modulating CD32B activity, in particular modulating the inhibitory signals associated with the CD32B signaling pathway, e.g., inhibiting cell proliferation and/or inhibiting antibody production. In yet other embodiments, because CD32B is also expressed by B cells, administration of therapies comprising CD32B binding agents may act on the plasma cell precursors, inhibiting differentiation and/or B cell activation and preventing transformation into a malignant plasma cell. In certain embodiments, the CD32B binding agents are anti-CD32B antibodies, analogs, derivatives, or fragments thereof.

The methods of the invention encompass the use of CD32B binding agents alone or in combination with other therapeutics known in the art to prevent, inhibit or reduce the growth of malignant cell, primary tumors or metastasis of cancerous cells. In certain embodiments, the binding agents, e.g. anti-CD32B antibodies, analogs, derivatives, or fragments thereof, used in the methods of the invention can be used in combination with other known cancer immunotherapies. The invention also encompasses the use of the CD32B binding agents of the invention in combination with other therapies known (e.g., chemotherapy and radiotherapy) in the art for the prevention and/or treatment of plasma cell disorders. In certain embodiments, the invention encompasses the use of anti-CD32B antibodies in combination with other antibodies known in the art for the treatment and or prevention of plasma cell disorders and/or B cell disorders. For example, the methods of the invention also encompass the use of, for example, Rituxin (target: CD20), Campath (target: CD52), Mylotarge (target: CD33), and Zevalin (target: CD20).

Accordingly, the invention provides methods of treating, preventing, managing or ameliorating a plasma cell disorder associated with CD32B expression, or one or more symptoms thereof. In a specific embodiment, a CD32B binding agent, e.g., an antibody, analog, derivative, or fragment thereof, used in the methods of the invention, when administered alone or in combination with a cytotoxic therapeutic antibody, inhibits or reduces the growth or metastasis of malignant cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth or metastasis in absence of said binding agent used in the methods of the invention. In another embodiment, a CD32B binding agent, e.g., an antibody, analog, derivative, or fragment thereof, used in the methods of the invention, when administered alone or in combination with a cytotoxic therapeutic antibody, inhibits or reduces the proliferation of malignant plasma cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the proliferation of said cells in the absence of said binding agent used in the methods of the invention. In other embodiments, a CD32B binding agent, e.g., an antibody, analog, derivative, or fragment thereof, used in the methods of the invention, when administered alone or in combination with a cytotoxic therapeutic antibody, inhibits or reduces the concentration of serum M protein is a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the concentration of serum M protein in said subject absence of said binding agent used in the methods of the invention. In still other embodiment, a CD32B binding agent, e.g., an antibody, analog, derivative, or fragment thereof, used in the methods of the invention, when administered alone or in combination with a cytotoxic therapeutic antibody, increases the hemoglobin value of a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the hemoglobin value of said subject in absence of said binding agent used in the methods of the invention. In other embodiments, a CD32B binding agent, e.g., an antibody, analog, derivative, or fragment thereof, used in the methods of the invention, when administered alone or in combination with a cytotoxic therapeutic antibody, reduces the total number and/or severity of one or more lytic bone lesions in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to that of said subject in the absence of said binding agent used in the methods of the invention. In other embodiments, a CD32B binding agent, e.g., an antibody, analog, derivative, or fragment thereof, used in the methods of the invention, when administered alone or in combination with a cytotoxic therapeutic antibody, reduces the concentration of serum calcium in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the concentration of serum calcium in said subject in the absence of said binding agent used in the methods of the invention.

The invention further provides methods of treating a plasma cell disorder, or one or more symptoms thereof, in subjects with early stage disease to slow or prevent the progression of the disease, to slow or prevent the damage to tissues or organs from the accumulation of amyloid protein, and/or to reduce the need for other therapy. In specific embodiments, the methods of the invention prevent or slow the progression of said plasma cell disorder to a more aggressive disease state. For example, treatment of a subject according to the methods of the invention may prevent or delay the progression of multiple myeloma (MM) from an asymptomatic state, e.g., monoclonal gammopathies of undetermined significance (MGUS), smoldering multiple myeloma (SMM), indolent multiple myeloma or early stage MM (stage I), to a later stage MM (stage II or stage III) in a subject by 2 months, 4 months, 6 months, 8, months, 10 months, 12 months, 15 months, 18 months, 21 months, 24 months, 2.5 years, 3 years, 4 years, 5 years, 6 years, 8 years, or 10 years or longer relative to a subject with similar clinical parameters who did not receive treatment. In a specific embodiment, the treatment of a subject according to the methods of the invention may prevent or delay the progression of gammopathies of undetermined significance (MGUS) to multiple myeloma (MM) in a subject by 2 months, 4 months, 6 months, 8, months, 10 months, 12 months, 15 months, 18 months, 21 months, 24 months, 2.5 years, 3 years, 4 years, 5 years, 6 years, 8 years, or 10 years or longer relative to a subject with similar clinical parameters who did not receive treatment. In another embodiment, the treatment of a subject according to the methods of the invention may prevent or delay the progression of multiple myeloma (MM) to plasma cell leukemia (PCL) in a subject by 2 months, 4 months, 6 months, 8, months, 10 months, 12 months, 15 months, 18 months, 21 months, 24 months, 2.5 years, 3 years, 4 years, 5 years, 6 years, 8 years, or 10 years or longer relative to a subject with similar clinical parameters who did not receive treatment.

Standard cancer therapies and their dosages, routes of administration and recommended usage are known in the art and have been described in the literature, see, e.g., Physician's Desk Reference (56^{th} ed., 2002, which is incorporated herein by reference).

5.1.1 Diagnostic and Prognostic Methods

In addition to therapeutic or prophylactic methods, the invention also encompasses methods for the diagnosis of plasma cell disorders. Currently, standard methods of diagnosis rely on recognition of symptoms of the disease rather than a recognition of the underlying cause of the disease itself. Accurate diagnosis of a plasma cell disorder, to the exclusion of other pathologies, would lead to improved treatment protocols. For example, amyloidosis is normally diagnosed based on discovery of amyloid deposits in organs or other body tissues. While analysis of blood, urine, bone marrow, or the amyloid deposits themselves is of use, because systemic amyloidosis can be caused by a number of conditions (see, e.g., Dobson, 2006, 13:219-227, hereby incorporated by reference in its entirety), diagnosis, and therefore the decision of an effective treatment strategy, can be problematic. Similarly, diagnosis and treatment of MM is based on a staging of the disease according to defined set of clinical parameters including presence of serum M protein, involvement of marrow, and presence/absence of anemia (see, e.g., Mihou et al., 2006, Haematologica 91:1149-1150, hereby incorporated by reference in its entirety). However, not all staging systems are equivalent, and the choice of a particular staging system may affect the choice or course of treatment plan (see, e.g., Kim et al., 2006, Biol. Blood Marrow Transplant. 12:837-844 and Rajikumar et al., 2002, Mayo Clin. Proc. 77:814, hereby incorporated by reference in their entireties).

The instant invention encompasses methods of diagnosing plasma cell disorders that rely on the molecular characterization of a subject's plasma cells and, in particular, the characterization of surface expression of CD32B on said cells. In certain embodiments, plasma cells are characterized by the presence of CD138. As set forth in Section 6.1, the Applicants have discovered that the surface expression of CD32B on plasma cells, in particular CD138 positive cells, correlates with both plasma cell disease and the aggressivity of said disease. In certain embodiments, a decreased expression of CD32B in plasma cells relative to a standard level or to that in a population of non-malignant plasma cells correlates with a plasma cell disorder. In other embodiments, the expression of CD32B of plasma cells is inversely proportional to the aggressivity of the plasma cell disorder. In related embodiments, the plasma cell disorder is multiple myeloma. In particular, the methods of the invention comprise the use of CD32B binding agents to characterize the CD32B expression of a subject's plasma cells. In certain embodiments, the CD32B binding agent is an anti-CD32B antibody, analog, derivative, or fragment thereof and the plasma cells are characterized by the expression of CD138 (e.g., bind to anti-CD138 specific antibodies).

Certain plasma cell disorders are characterized as “asymptomatic” in that signs of the disease are noted during routine procedures, e.g., physical exams, and in that the disease, initially, causes no adverse effects to the subject. Such asymptomatic plasma cell disorders (e.g., monoclonal gammopathies of undetermined significance (MGUS), smoldering multiple myeloma (SMM), indolent multiple myeloma) and some early stage MM (stage 1) are typically not treated, but only monitored for signs of disease progression. Asymptomatic plasma cell disorders can remain static and not progress for a number of years, especially in the case of MGUS, and thus treatment is delayed until necessary due to the adverse side-effects of current therapies, e.g., chemotherapy. Such a “wait and see” approach has potentially adverse consequences, allowing malignant disease to become established in between monitoring and/or prior to noticeable symptoms. As discussed supra and in Section 6.1, Applicants have discovered that the CD32B expression of the plasma cells of a subject correlates with disease and disease state, e.g., aggressivity. Thus, the present invention provides methods of monitoring and/or assessing a plasma cell disorder based on a cell surface protein of the disease itself rather than on disease symptom. In certain embodiments, the invention provides for a method for monitoring the progression or the development of a plasma cell disorder in a subject comprising characterizing the CD32B expression of the plasma cells of said subject. In certain embodiments, the level of CD32B expression of a subjects plasma cells is compared to a standard level, to the level of a population of non-malignant plasma cells, or to a prior level determined for said subject. In certain embodiments, plasma cells are identified by CD138 expression. In other embodiments, the invention provides methods for monitoring the progression or the development of a plasma cell disorder in a subject comprising characterizing the CD32B expression of the plasma cells of said subject, wherein the treatment of said disorder is altered based on said level of CD32B expression. In certain embodiments, the subject requiring monitoring for progression or development of a plasma cell disorder has been diagnosed with monoclonal gammopathy of undetermined significance (MGUS). In other embodiments, the subject requiring monitoring for progression or development of a plasma cell disorder have been diagnosed with asymptomatic multiple myeloma (MM). In other embodiments, the subject diagnosed with multiple myeloma (MM) is monitored for progression of the disease comprising characterizing the CD32B expression of the plasma cells of said subject. In certain embodiments, the diagnosis of multiple myeloma (MM) or light-chain amyloidosis is based on a characterization, recognition or finding of clonal plasma cells. In related embodiments, the clonal plasma cells are characterized by the expression of κ light chain. In other embodiments, the clonal plasma cells are characterized by the expression of λ light chains. In certain embodiments, the level of CD32B expression of the plasma cells of a subject in need thereof may be determined at intervals of 1 week, 2 weeks, 1 month, 2 months, 4 months, 6 months, 8 months, 12 months, 16 months, 20 months, 24 months, 3 years or 5 or more years.

Binding of an anti-CD32B antibody to a sample comprising plasma cells or plasma cell lysates may be determined, for example, using immunological or biochemical based methods including, but not limited to, an ELISA assay, immunoprecipitation assay, affinity chromatography, fluorescence activated cell sorting (FACS), and equilibrium dialysis. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety).

5.2 Autoimmune Disease

The methods of the invention may be used to treat, prevent, manage, or ameliorate an autoimmune disease, or one of more symptoms thereof. The present invention provides methods of preventing, treating, or managing one or more symptoms associated with an autoimmune disorder in a subject, comprising administering to said subject a therapeutically effective amount of the CD32B binding agent of the invention, e.g., a humanized anti-CD32B antibody, analog, derivative, or fragment thereof. The invention also provides methods for preventing, treating, or managing one or more symptoms associated with an autoimmune disease further comprising, administering to said subject a therapeutically effective amount of one or more immunomodulatory agents, including antibodies, known in the art for treatment of an autoimmune disorder or symptom thereof. For example, but not by way of limitation, the CD32B binding agents of the invention, e.g., humanized anti-CD32B antibodies, analogs, derivatives, or fragments thereof, can be used in combination with Orthoclone OKT3, ReoPro, Zenapax, Simulec, Rituximab, Synagis, and Remicade.

Examples of autoimmune disorders that may be treated by administering the antibodies of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis herpetiformis vasculitis, vitiligo, and Wegener's granulomatosis. As is known in the art, some autoimmune disorders are associated with an inflammatory condition and, thus, there is overlap between what is considered an autoimmune disorder and an inflammatory disorder. Therefore, some autoimmune disorders may also be characterized as inflammatory disorders. Such inflammatory disorders have etiologies, in part, due to auto-reactive antibodies. Examples of inflammatory disorders which can be prevented, treated or managed in accordance with the methods of the invention include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacteria infections.

In certain embodiments of the invention, the CD32B binding agents of the invention, e.g., humanized anti-CD32B antibodies, analogs, derivatives, or fragments thereof, may be used to treat an autoimmune disease that is more prevalent in one sex. For example, the prevalence of Graves' disease in women has been associated with expression of Fc□RIIB2 (see Estienne et al., 2002, FASEB J. 16:1087-1092). Accordingly, CD32B binding agents of the invention may be used to treat, prevent, ameliorate, or manage Graves' disease.

CD32B binding agents of the invention, e.g., humanized anti-CD32B antibodies, analogs, derivatives, or fragments thereof, can also be used to reduce the inflammation experienced by animals, particularly mammals, with inflammatory disorders. In a specific embodiment, an antibody reduces the inflammation in an animal by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the inflammation in an animal in the not administered said antibody. In another embodiment, a combination of antibodies reduce the inflammation in an animal by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the inflammation in an animal in not administered said antibodies.

Humanized antibodies of the invention can also be used to prevent the rejection of transplants.

5.3 CD32B-Specific Antibodies, Analogs, Derivatives, and Antigen-Binding Fragments Thereof

The present invention encompasses the use of a CD32B-specific antibody, an analog, derivative or an antigen-binding fragment thereof (e.g., one or more complementarity determining regions (“CDRs”) of a CD32B-specific antibody) in the prevention, treatment, management or amelioration of a plasma cell disorder, or one or more symptoms thereof. In a specific embodiment, the present invention encompasses the use of antibodies (preferably monoclonal antibodies) or fragments thereof that specifically bind FcγRIIB, preferably human FcγRIIB, more preferably native human FcγRIIB with a greater affinity than said antibodies or fragments thereof bind FcγRIIA, preferably human FcγRIIA, more preferably native human FcγRIIA. Representative antibodies are disclosed in U.S. Patent Application Publications US 2005/02157667, US 2004/0185045, US 2005/0260213, and US 2006/0013810; International Patent Application Publications WO 2005/110474 and WO 2005/115452; and U.S. Provisional Patent Application Publication No. 60/816,126 filed Jun. 23, 2006, herein expressly incorporated by reference in their entireties. Preferably, the antibodies bind the extracellular domain of native human FcγRIIB. In certain embodiments, the antibodies or fragments thereof bind to FcγRIIB with an affinity greater than two-fold, four fold, 6 fold, 10 fold, 20 fold, 50 fold, 100 fold, 1000 fold, 10⁴ fold, 10⁵ fold, 10⁶ fold, 10⁷ fold, or 10⁸ fold than said antibodies or fragments thereof bind FcγRIIA. In one particular embodiment, the antibody is or is derived from monoclonal antibody produced by clones 2B6 or 3H7 which bind FcγRIIB, having ATCC accession numbers PTA-4591 or PTA-4592, respectively, or a monoclonal antibody produced by clones 1D5, 2E1, 2H9, 2D11, and 1F2 having ATCC Accession numbers, PTA-5958, PTA-5961, PTA-5962, PTA-5960, and PTA-5959, respectively. In yet other embodiments the invention encompassed a humanized version of antibody 2B6, encoded by plasmid pMGx0675, having ATCC accession number PTA-7609. Plasmid pMGx0675 includes the nucleotide sequences SEQ ID NO:1 and SEQ ID NO:2 that encode the heavy chain amino acid sequence SEQ ID NO:3 and the light chain amino acid sequence SEQ ID NO:4, respectively. Hybridomas producing antibodies 2B6 and 3H7, and the plasmid producing pMGx0675 have been deposited with the American Type Culture Collection (10801 University Blvd., Manassas, Va. 20110-2209) on Aug. 13, 2002 (for hybridomas producing 2B6 and 3H7) and May 23, 2006 (for plasmid pMGx0675) under the provisions of the Budapest Treaty on the International Recognition of the Deposit of Microorganisms for the Purposes of Patent Procedures and are incorporated herein by reference in their entirety. In preferred embodiments, the antibodies described above are human antibodies or humanized antibodies.

In yet other embodiments, the invention encompasses the use of FcγRIIB antibodies that bind exclusively to FcγRIIB and have no affinity for FcγRIIA via the variable domain using standard methods known in the art and disclosed herein. In a preferred embodiment, the antibodies are human or humanized.

In a specific embodiment, the invention encompasses an antibody, preferably a humanized antibody, comprising the CDRs of 2B6 or of 3H7. In particular, the invention encompasses a humanized antibody with the heavy chain variable domain having the amino acid sequence of SEQ ID NO:5, SEQ ID NO:6 or SEQ ID NO:7 and the light chain variable domain having the amino acid sequence of SEQ ID NO:8, SEQ ID NO:9, SEQ ID NO:10, SEQ ID NO: 11. In a specific embodiment, the invention encompasses a humanized antibody with the heavy chain variable domain having the amino acid sequence of SEQ ID NO:12 and the light chain variable domain having the amino acid sequence of SEQ ID NO:13. In a preferred embodiment, the invention encompasses a humanized antibody with the heavy chain variable domain having the amino acid sequence of SEQ ID NO:7 and the light chain variable domain having the amino acid sequence of SEQ ID NO: 11. In yet another preferred embodiment, the humanized antibodies of the invention further do not bind Fc activation receptors, e.g., FcγRIIIA, FcγRIIIB, etc via the Fc domain.

The constant domains of the antibodies may be selected with respect to the proposed function of the antibody, in particular with regard to the effector function which may be required. In some embodiments, the constant domains of the antibodies are human IgA, IgE, IgG or IgM domains. In preferred embodiments, the antibodies are humanized antibodies. In a specific embodiment, human IgG constant domains, especially of the IgG1 and IgG3 isotypes are used, especially when humanized antibodies are intended for therapeutic uses and antibody effector functions are needed. In alternative embodiments, IgG2 and IgG4 isotypes are used when the humanized antibody of is intended for therapeutic purposes and antibody effector function is not required. In other embodiments, the invention encompasses the use of humanized antibodies comprising one or more amino acid modifications in the Fc region such as those disclosed in U.S. Patent Application Publication Nos. US 2005/0037000, US 2005/0064514; International Patent Application Publication Nos. WO 2004/063551; U.S. patent application Ser. No. 11/271,140 filed Nov. 10, 2005; U.S. Provisional Application Nos. 60/439,498; 60/456,041; 60/514,549; 60/587,251; 60/636,056; 60/626,510; 60/707,419, and 60/781,564 filed on Jan. 9, 2003; Mar. 19, 2003, Oct. 23, 2003, Jul. 12, 2004, Dec. 13, 2004, Nov. 10, 2004, Aug. 10, 2005, and Mar. 10, 2006 respectively; and U.S. Pat. Nos. 5,624,821 and 5,648,260 and European Patent No. EP 0 307 434; all of which are incorporated herein by reference in their entireties.

The antibodies, preferably humanized antibodies, of the invention bind the extracellular domain of native human FcγRIIB via their variable domain. The anti-FcγRIIB antibodies, preferably humanized antibodies, of the invention may have a heavy chain variable region comprising the amino acid sequence of CDR1 (SEQ ID NO:14, SEQ ID NO:15, an amino acid sequence corresponding to amino acids 31-35 as set forth in SEQ ID NO:6, or an amino acid sequence corresponding to amino acids 31-35 as set forth in SEQ ID NO:7) and/or CDR2 (SEQ ID NO:16, SEQ ID NO:17, an amino acid sequence corresponding to amino acids 50-66 as set forth in SEQ ID NO:6, or an amino acid sequence corresponding to amino acids 50-66 as set forth in SEQ ID NO:7) and/or CDR3 (SEQ ID NO:18, SEQ ID NO:19, an amino acid sequence corresponding to amino acids 100-111 as set forth in SEQ ID NO:6, or an amino acid sequence corresponding to amino acids 100-111 as set forth in SEQ ID NO:7) and/or a light chain variable region comprising the amino acid sequence of CDR1 (SEQ ID NO:20, SEQ ID NO:21, or an amino acid sequence corresponding to amino acids 24-34 as set forth in SEQ ID NO:1) and/or a CDR2 (SEQ ID NO:22, SEQ ID NO:23, SEQ ID NO:24, SEQ ID NO:25, or an amino acid sequence corresponding to amino acids 50-56 as set forth in SEQ ID NO:11) and/or CDR3 (SEQ ID NO:26, SEQ ID NO:27, or an amino acid sequence corresponding to amino acids 90-98 as set forth in SEQ ID NO:11).

In one specific embodiment, the invention provides a humanized 2B6 antibody, wherein the VH region consists of the FR segments from the human germline VH segment VH1-18 (Matsuda et al., 1998, J. Exp. Med. 188:2151062) and JH6 (Ravetch et al., 1981, Cell 27 (3 Pt. 2): 583-91), and one or more CDR regions of the 2B6 VH, having the amino acid sequence of SEQ ID NO:14, SEQ ID NO:16, or SEQ ID NO:18. In specific embodiments, the 2B6 VH has the amino acid sequence of SEQ ID NO:5, SEQ ID NO:6, or SEQ ID NO:28. In another specific embodiment, the humanized 2B6 antibody further comprises a VL region, which consists of the FR segments of the human germline VL segment VK-A26 (Lautner-Rieske et al., 1992, Eur. J. Immunol. 22:1023-1029) and JK4 (Hieter et al., 1982, J. Biol. Chem. 257:1516-22), and one or more CDR regions of 2B6 VL, having the amino acid sequence of SEQ ID NO:20, SEQ ID NO:22, SEQ ID NO:23, SEQ ID NO:24, and SEQ ID NO:26. In one embodiment, the 2B6 VL has the amino acid sequence of SEQ ID NO:8; SEQ ID NO:9, SEQ ID NO:10, or SEQ ID NO:11. In a preferred embodiment, the 2B6 antibody of the invention comprises a VL comprising the amino acid sequence SEQ ID NO:11 and a VH comprising the amino acid sequence SEQ ID NO:7.

In another specific embodiment, the invention provides a humanized 3H7 antibody, wherein the VH region consists of the FR segments from a human germline VH segment and the CDR regions of the 3H7 VH, having the amino acid sequence of SEQ ID NO:12. In another specific embodiment, the humanized 3H7 antibody further comprises a VL regions, which consists of the FR segments of a human germline VL segment and the CDR regions of 3H7 VL, having the amino acid sequence of SEQ ID NO: 13.

The invention also encompasses the use of other antibodies or fragments thereof that specifically bind Fc□RIIB, preferably human Fc□RIIB, more preferably native human Fc□RIIB, that are derived from clones including but not limited to 1D5, 2E1, 2H9, 2D11, and 1F2 having ATCC Accession numbers, PTA-5958, PTA-5961, PTA-5962, PTA-5960, and PTA-5959 respectively. Hybridomas producing the above-identified clones were deposited under the provisions of the Budapest Treaty with the American Type Culture Collection (10801 University Blvd., Manassas, Va. 20110-2209) on May 7, 2004, and are incorporated herein by reference. In a preferred embodiments, the antibodies described above are humanized. In some embodiments, the humanized antibody of the invention contains both the light chain as well as at least the variable domain of a heavy chain. In some embodiments, the constant domain is a complement fixing constant domain where it is desired that the humanized antibody exhibit cytotoxic activity, and the class is typically IgG₁. In other embodiments, where such cytotoxic activity is not desirable, the constant domain may be of the IgG₂ class. The humanized antibody of the invention may comprise sequences from more than one class or isotype, and selecting particular constant domains to optimize desired effector functions is within the ordinary skill in the art.

In one embodiment, the antibodies of the invention, or fragments thereof, modulate at least one activity of FcγRIIB. In one embodiment, said activity is activation of B cell receptor-mediated signaling. In a particular embodiment, the antibodies of the invention modulate B cell activity, B cell proliferation, antibody production, intracellular calcium influx, or activity of one or more downstream signaling molecules in the FcγRIIB signal transduction pathway. In yet another particular embodiment, the antibodies of the invention modulate phosphorylation of FcγRIIB or SHIP recruitment. In a further embodiment of the invention, the antibodies modulate MAP kinase activity or Akt recruitment in the B cell receptor mediated signaling pathway. In another embodiment, the antibodies of the invention modulate FcγRIIB-mediated inhibition of FcεRI signaling. In a particular embodiment, the antibodies of the invention modulate FcεRI-induced mast cell activation, calcium mobilization, degranulation, cytokine production, or serotonin release. In another embodiment, the antibodies of the invention modulate phosphorylation of FcγRIIB, modulate recruitment of SHIP, modulate SHIP phosphorylation and its association with Shc, or modulate activation of MAP kinase family members (e.g., Erk1, Erk2, JNK, p38, etc.). In yet another embodiment, the antibodies of the invention modulate tyrosine phosphorylation of p62dok and its association with SHIP and rasGAP. In another embodiment, the antibodies of the invention modulate FcγR-mediated phagocytosis in monocytes or macrophages. In another embodiment, the antibodies of the invention modulate phagocytosis, and clearance of opsonized particles by splenic macrophages. In certain embodiments, the anti-CD32B antibodies, analogs, derivatives or fragments thereof agonize CD32B activity. In other embodiments, the anti-CD32B antibodies, analogs, derivatives or fragments thereof antagonize CD32B activity.

Antibodies used in the methods of the invention include, but are not limited to, monoclonal antibodies, synthetic antibodies, recombinantly produced antibodies, multispecific antibodies, human antibodies, chimeric antibodies, camelized antibodies, single-chain Fvs (scFv), single chain antibodies, Fab fragments, F(ab′) fragments, disulfide-linked Fvs (sdFv), intrabodies, and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to FcγRIIB with greater affinity than said immunoglobulin molecule binds FcγRIIA.

The antibodies used in the methods of the invention may be from any animal origin including birds and mammals (e.g., human, non-human primate, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or libraries of synthetic human immunoglobulin coding sequences or from mice that express antibodies from human genes.

The antibodies used in the methods of the present invention may be monospecific, bispecific, trispecific or of greater multispecificity. Multispecific antibodies may immunospecifically bind to different epitopes of FcγRIIB or immunospecifically bind to both an epitope of FcγRIIB as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553; Todorovska et al., 2001 Journal of Immunological Methods, 248:47-66. In particular embodiments, the antibodies of the invention are multispecific with specificities for FcγRIIB and for a cancer antigen or any other cell surface marker specific for a cell designed to be killed, e.g., in treating or preventing a particular disease or disorder, or for other Fc receptors, e.g., FcγRIIIA, FcγRIIIB, etc.

In a specific embodiment, an antibody used in the methods of the present invention is an antibody or an antigen-binding fragment thereof (e.g., comprising one or more complementarily determining regions (CDRs), preferably all 6 CDRs) of the antibody produced by clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively (e.g., the heavy chain CDR3). In another embodiment, an antibody used in the methods of the present invention binds to the same epitope as the mouse monoclonal antibody produced from clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively and/or competes with the mouse monoclonal antibody produced from clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively as determined, e.g., in an ELISA assay or other appropriate competitive immunoassay, and also binds FcγRIIB with a greater affinity than said antibody or a fragment thereof binds FcγRIIA.

The antibodies used in the methods of the invention include derivatives that are modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

For some uses, including in vivo use of antibodies in humans and in vitro detection assays, it may be preferable to use human, chimeric or humanized antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Further, the antibodies of the invention can, in turn, be utilized to generate anti-idiotype antibodies using techniques well known to those skilled in the art. (See, e.g., Greenspan & Bona, 1989, FASEB J. 7:437-444; and Nissinoff, 1991, J. Immunol. 147:2429-2438). The invention provides methods employing the use of polynucleotides comprising a nucleotide sequence encoding an antibody of the invention or a fragment thereof.

The present invention encompasses the use of single domain antibodies, including camelized single domain antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties). In one embodiment, the present invention provides single domain antibodies comprising two VH domains with modifications such that single domain antibodies are formed.

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor. The humanized antibodies of the invention may be engineered by methods described in Ward et al. to increase biological half-lives (See U.S. Pat. No. 6,277,375 B1). For example, humanized antibodies of the invention may be engineered in the Fc-hinge domain to have increased in vivo or serum half-lives.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies used in the methods of the invention may also be modified by the methods and coupling agents described by Davis et al. (See U.S. Pat. No. 4,179,337) in order to provide compositions that can be injected into the mammalian circulatory system with substantially no immunogenic response.

The present invention also encompasses the use of antibodies or antibody fragments comprising the amino acid sequence of any of the antibodies of the invention with mutations (e.g., one or more amino acid substitutions) in the framework or CDR regions. Preferably, mutations in these humanized antibodies maintain or enhance the avidity and/or affinity of the antibodies for CD32B to which they immunospecifically bind. Standard techniques known to those skilled in the art (e.g., immunoassays) can be used to assay the affinity of an antibody for a particular antigen.

The invention encompasses modification of framework residues of the antibodies of the invention. Framework residues in the framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated herein by reference in their entireties.)

The invention also provides antibodies with altered oligosaccharide content. Oligosaccharides, as used herein, refer to carbohydrates containing two or more simple sugars and the two terms may be used interchangeably herein. Carbohydrate moieties of the instant invention will be described with reference to commonly used nomenclature in the art. For a review of carbohydrate chemistry, see, e.g., Hubbard et al., 1981 Ann. Rev. Biochem., 50: 555-583, which is incorporated herein by reference in its entirety. This nomenclature includes, for example, Man which represents mannose; GlcNAc which represents 2-N-acetylglucosamine; Gal which represents galactose; Fuc for fucose and Glc for glucose. Sialic acids are described by the shorthand notation NeuNAc for 5-N-acetylneuraminic acid, and NeuNGc for 5-glycolneuraminic.

In general, antibodies contain carbohydrate moieties at conserved positions in the constant region of the heavy chain, and up to 30% of human IgGs have a glycosylated Fab region. IgG has a single N-linked biantennary carbohydrate structure at Asn 297 which resides in the CH2 domain (Jefferis et al., 1998, Immunol. Rev. 163: 59-76; Wright et al., 1997, Trends Biotech 15: 26-32). Human IgG typically has a carbohydrate of the following structure; GlcNAc(Fucose)-GlcNAc-Man-(ManGlcNAc)₂. However variations among IgGs in carbohydrate content does occur which leads to altered function, see, e.g., Jassal et al., 2001 Biochem. Biophys. Res. Commun. 288: 243-9; Groenink et al., 1996 J. Immunol. 26: 1404-7; Boyd et al., 1995 Mol. Immunol. 32: 1311-8; Kumpel et al., 1994, Human Antibody Hybridomas, 5: 143-51. The invention encompasses antibodies comprising a variation in the carbohydrate moiety that is attached to Asn 297. In one embodiment, the carbohydrate moiety has a galactose and/or galactose-sialic acid at one or both of the terminal GlcNAc and/or a third GlcNac arm (bisecting GlcNAc).

In some embodiments, the antibodies used in the methods of the invention are substantially free of one or more selected sugar groups, e.g., one or more sialic acid residues, one or more galactose residues, one or more fucose residues. An antibody that is substantially free of one or more selected sugar groups may be prepared using common methods known to one skilled in the art, including, for example, recombinantly producing an antibody of the invention in a host cell that is defective in the addition of the selected sugar groups(s) to the carbohydrate moiety of the antibody, such that about 90-100% of the antibody in the composition lacks the selected sugar group(s) attached to the carbohydrate moiety. Alternative methods for preparing such antibodies include, for example, culturing cells under conditions which prevent or reduce the addition of one or more selected sugar groups, or post-translational removal of one or more selected sugar groups.

In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields R. L. et al., 2001, J. Biol. Chem. 277(30): 26733-40; Davies J. et al., 2001, Biotechnology & Bioengineering, 74(4): 288-294). In another specific embodiment, the altered carbohydrate modifications enhance the binding of antibodies of the invention to Fc□RIIB receptor. Altering carbohydrate modifications in accordance with the methods of the invention includes, for example, increasing the carbohydrate content of the antibody or decreasing the carbohydrate content of the antibody. Methods of altering carbohydrate contents are known to those skilled in the art, see, e.g., Wallick et al., 1988, Journal of Exp. Med. 168(3): 1099-1109; Tao et al., 1989 Journal of Immunology, 143(8): 2595-2601; Routledge et al., 1995 Transplantation, 60(8): 847-53; Elliott et al. 2003; Nature Biotechnology, 21: 414-21; Shields et al. 2002 Journal of Biological Chemistry, 277(30): 26733-40; all of which are incorporated herein by reference in their entirety.

In some embodiments, the invention encompasses the use antibodies comprising one or more glycosylation sites, so that one or more carbohydrate moieties are covalently attached to the antibody. In other embodiments, the invention encompasses antibodies comprising one or more glycosylation sites and one or more modifications in the Fc region, such as those disclosed supra and those known to one skilled in the art. In preferred embodiments, the one or more modifications in the Fc region enhance the affinity of the antibody for an activating Fc□R, e.g., Fc□RIIIA, relative to the antibody comprising the wild type Fc regions. Antibodies of the invention with one or more glycosylation sites and/or one or more modifications in the Fc region have an enhanced antibody mediated effector function, e.g., enhanced ADCC activity. In some embodiments, the invention further comprises antibodies comprising one or more modifications of amino acids that are directly or indirectly known to interact with a carbohydrate moiety of the antibody, including, but not limited to, amino acids at positions 241, 243, 244, 245, 245, 249, 256, 258, 260, 262, 264, 265, 296, 299, and 301. Amino acids that directly or indirectly interact with a carbohydrate moiety of an antibody are known in the art, see, e.g., Jefferis et al., 1995 Immunology Letters, 44: 111-7, which is incorporated herein by reference in its entirety.

The invention encompasses the use of antibodies that have been modified by introducing one or more glycosylation sites into one or more sites of the antibodies, preferably without altering the functionality of the antibody, e.g., binding activity to Fc□RIIB. Glycosylation sites may be introduced into the variable and/or constant region of the antibodies of the invention. As used herein, “glycosylation sites” include any specific amino acid sequence in an antibody to which an oligosaccharide (i.e., carbohydrates containing two or more simple sugars linked together) will specifically and covalently attach. Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. The antibodies used in the methods of the invention may comprise one or more glycosylation sites, including N-linked and O-linked glycosylation sites. Any glycosylation site for N-linked or O-linked glycosylation known in the art may be used in accordance with the instant invention. An exemplary N-linked glycosylation site that is useful in accordance with the methods of the present invention, is the amino acid sequence: Asn-X-Thr/Ser, wherein X may be any amino acid and Thr/Ser indicates a threonine or a serine. Such a site or sites may be introduced into an antibody used in the methods of the invention using methods well known in the art to which this invention pertains. See, for example, “In Vitro Mutagenesis,” Recombinant DNA: A Short Course, J. D. Watson, et al. W.H. Freeman and Company, New York, 1983, chapter 8, pp. 106-116, which is incorporated herein by reference in its entirety. An exemplary method for introducing a glycosylation site into an antibody of the invention may comprise: modifying or mutating an amino acid sequence of the antibody so that the desired Asn-X-Thr/Ser sequence is obtained.

In some specific embodiments, the invention encompasses the use of modified Fc□RIIB antibodies wherein the N-glycosylation consensus site Asn₅₀-Val-Ser of the CDR2 region has been modified, so that the glycosylation site at position 50 is eliminated. Although not intending to be bound by a particular mechanism of action, removal of the glycosylation site may limit potential variation in production of the antibody as well as potential immunogenicity in a pharmaceutical application. In a specific embodiment, the invention encompasses the use of a humanized Fc□RIIB antibody wherein the amino acid at position 50 has been modified, e.g., deleted or substituted. In another specific embodiment, the invention further encompasses the use of an antibody with an amino acid modification, e.g., deletion or substitution, at position 51. In one specific embodiment, the invention encompasses the use of a humanized Fc□RIIB antibody wherein the amino acid at position 50 has been replaced with tyrosine. In another more specific embodiment, the invention encompasses the use of a Fc□RIIB antibody wherein the amino acid at position 50 has been replaced with tyrosine and the amino acid at position 51 has been replaced with alanine.

Standard techniques known to those skilled in the art can be used to introduce mutations in the nucleotide sequence encoding an antibody, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original antibody or fragment thereof. In a preferred embodiment, the derivatives have conservative amino acid substitutions made at one or more predicted non-essential amino acid residues.

The present invention also encompasses antibodies or fragments thereof comprising an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of the mouse monoclonal antibody produced by clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively. In preferred embodiments of the invention, the antibody or fragment thereof comprises a heavy chain having the amino acid sequence SEQ ID NO:3 and/or light chain having the amino acid sequence SEQ ID NO:4. The present invention further encompasses antibodies or fragments thereof that specifically bind FcγRIIB with greater affinity than said antibody or fragment thereof binds FcγRIIA and/or bind to FcγRIIB and block the Fc binding domain of FcγRIIB, said antibodies or antibody fragments comprising an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of the mouse monoclonal antibody produced by clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively. The determination of percent identity of two amino acid sequences can be determined by any method known to one skilled in the art, including BLAST protein searches.

The present invention also encompasses the use of antibodies or antibody fragments that specifically bind FcγRIIB with greater affinity than said antibodies or fragments thereof binds FcγRIIA and/or bind to FcγRIIB and block the Fc binding domain of FcγRIIB, wherein said antibodies or antibody fragments are encoded by a nucleotide sequence that hybridizes to the nucleotide sequence of the mouse monoclonal antibody produced by clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively, under stringent conditions. In a specific embodiments, the invention encompasses the use of antibodies or antibody fragments that specifically bind FcγRIIB with greater affinity than said antibodies or fragments thereof binds FcγRIIA and/or bind to FcγRIIB and block the Fc binding domain of FcγRIIB, wherein said antibodies or antibody fragments are encoded by a nucleotide sequence that hybridizes to the nucleotide sequence encoding the heavy chain variable domain or the heavy chain of 2B6 v3.5 Fc088, e.g., SEQ ID NO:29 or SEQ ID NO:1, respectively, under stringent conditions. In yet other embodiments, the invention encompasses the use of antibodies or antibody fragments that specifically bind FcγRIIB with greater affinity than said antibodies or fragments thereof binds FcγRIIA and/or bind to FcγRIIB and block the Fc binding domain of FcγRIIB, wherein said antibodies or antibody fragments are encoded a nucleotide sequence that hybridizes to the nucleotide sequence encoding the light chain variable domain or the light chain of 2B6 v3.5 Fc088, e.g., SEQ ID NO:30 or SEQ ID NO:2, respectively, under stringent conditions. In a preferred embodiment, the invention provides antibodies or fragments thereof that specifically bind FcγRIIB with greater affinity than said antibodies or fragments thereof bind FcγRIIA, said antibodies or antibody fragments comprising a variable light chain and/or variable heavy chain encoded by a nucleotide sequence that hybridizes under stringent conditions to the nucleotide sequence of the variable light chain and/or variable heavy chain of the mouse monoclonal antibody produced by clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively, under stringent conditions. In another preferred embodiment, the invention provides antibodies or fragments thereof that specifically bind FcγRIIB with greater affinity than said antibodies or fragments thereof bind FcγRIIA, said antibodies or antibody fragments comprising one or more CDRs encoded by a nucleotide sequence that hybridizes under stringent conditions to the nucleotide sequence of one or more CDRs of the mouse monoclonal antibody produced by clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively. Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3, incorporated herein by reference).

5.4 CD32B Agonists and Antagonists

In addition to the use of a CD32B-specific antibody, an analog, derivative, or an antigen-binding fragment thereof in the methods and compositions of the invention, other compounds that bind CD32, i.e., CD32B binding agents, and modulate CD32B activity, e.g., CD32B agonists or CD32B antagonists, may be used in accordance with the invention. Such compounds include, but are not limited to, proteinaceous molecules (e.g., proteins, polypeptides (e.g., soluble CD32B polypeptides), peptides, fusion proteins (e.g., soluble CD32B polypeptides conjugated to a therapeutic moiety), nucleic acid molecules (e.g., CD32B antisense nucleic acid molecules, triple helices, dsRNA that mediates RNAi, or nucleic acid molecules encoding proteinaceous molecules), organic molecules, inorganic molecules, small organic molecules, drugs, and small inorganic molecules. CD32B binding agents can be identified and characterized using techniques well-known in the art or described herein.

In certain embodiments, CD32B agonists increase or enhance a function, activity, and/or expression of a CD32B polypeptide in a subject with a plasma cell disorder. CD32B agonists may directly bind to a CD32B polypeptide and directly or indirectly modulate an activity and/or function of a plasma cell. In particular embodiments, CD32B agonists modulate plasma cell proliferation in a subject with a plasma cell disorder as determined by standard in vivo and/or in vitro assays described herein or well-known to those skilled in the art. In another embodiment, CD32B agonists or directly or indirectly modulate an activity and/or function of B-lymphocytes by utilizing antibody-dependent cytotoxicity (ADCC).

In other embodiments, CD32B antagonists reduce a function, activity, and/or expression of a CD32B polypeptide in a subject with a plasma cell disorder. In other embodiments, the CD32B antagonists directly bind to a CD32B polypeptide and directly or indirectly modulate an activity and/or function of a plasma cell. In particular embodiments, CD32B antagonists inhibit or reduce plasma cell proliferation in a subject with a plasma cell disorder as determined by standard in vivo and/or in vitro assays described herein or well-known to those skilled in the art. In a specific embodiment, CD32B antagonists mediate the depletion of lymphocytes, in particular peripheral blood cells of B-cell lineage, in a subject with a plasma cell disorder as determined by standard in vivo or in vitro assays described herein or well known to those of skill in the art. In another embodiment, CD32B agonists or directly or indirectly modulate an activity and/or function of lymphocytes of B-cell lineage by utilizing antibody-dependent cytotoxicity (ADCC).

In a preferred embodiment, proteins, polypeptides or peptides (including antibodies and fusion proteins) that are utilized as CD32B agonists or antagonists are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as CD32B agonists or antagonists are human or humanized.

Nucleic acid molecules encoding proteins, polypeptides, or peptides that modulate the function CD32B activity can be administered to a subject with a plasma cell disorder, in accordance with the methods of the invention. Further, nucleic acid molecules encoding derivatives, analogs, fragments or variants of proteins, polypeptides, or peptides that modulate CD32B activity can be administered to a subject with a plasma cell disorder in accordance with the methods of the invention. Preferably, such derivatives, analogs, variants and fragments retain activity of the full-length wild-type protein, polypeptide, or peptide.

5.5 CD32B Antibody Conjugates

The present invention encompasses the use of anti-CD32B antibodies recombinantly fused or chemically conjugated (including both covalently and non-covalently conjugations) to heterologous polypeptides (i.e., an unrelated polypeptide; or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. Antibodies may be used for example to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., PCT Publication No. WO 93/21232; EP 439,095; Naramura et al., 1994 Immunol. Lett., 39:91-99; U.S. Pat. No. 5,474,981; Gillies et al., 1992 Proc. Natl. Acad. Sci. USA, 89:1428-1432; and Fell et al., 1991, J. Immunol., 146:2446-2452, all of which are incorporated herein by reference in their entireties.

Further, an antibody may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin (i.e., PE-40), or diphtheria toxin, ricin, gelonin, and pokeweed antiviral protein, a protein such as tumor necrosis factor, interferons including, but not limited to, α-interferon (IFN-α), β-interferon (IFN-β), nerve growth factor (NGF), platelet derived growth factor (PDGF), tissue plasminogen activator (TPA), an apoptotic agent (e.g., TNF-α, TNF-β, AIM I as disclosed in PCT Publication No. WO 97/33899), AIM II (see, e.g., PCT Publication No. WO 97/34911), Fas Ligand (Takahashi et al., 1994 J. Immunol., 6:1567-1574), and VEGI (PCT Publication No. WO 99/23105), a thrombotic agent or an anti-angiogenic agent (e.g., angiostatin or endostatin), or a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), macrophage colony stimulating factor, (“M-CSF”), or a growth factor (e.g., growth hormone (“GH”); a protease, or a ribonuclease.

Antibodies can be fused to marker sequences, such as a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989 Proc. Natl. Acad. Sci. USA, 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984 Cell, 37:767) and the “flag” tag (Knappik et al., 1994 Biotechniques, 17(4):754-761).

The present invention further includes the use of compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to FcγRIIB may be recombined with one or more components, motifs, sections, parts, domains, fragments, etc. of one or more heterologous molecules.

The present invention also encompasses the use of antibodies conjugated to a diagnostic or therapeutic agent or any other molecule for which serum half-life is desired to be increased. The antibodies can be used diagnostically to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and acquorin; radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), Ianthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹ Pm), rhenium (¹⁸⁶Re, ¹⁸⁸Re), rhodium (¹⁰⁵Rh), ruthenium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

An antibody may be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating such therapeutic moieties to antibodies are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al., Immunol. Rev., 62:119-58, 1982.

An antibody or fragment thereof, with or without a therapeutic moiety conjugated to it, administered alone or in combination with cytotoxic factor(s) and/or cytokine(s) can be used as a therapeutic.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

5.6 Anti-Cancer Agents and Therapeutic Antibodies that Can be Used in Combination with a CD32B Binding Agents

In a specific embodiment, the methods of the invention encompass the administration of one or more angiogenesis inhibitors such as, but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); EGFr blockers/inhibitors (Iressa®, Tarceva®, Erbitux®, and ABX-EGF) Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Anti-cancer agents that can be used in combination with antibodies of the invention in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+mycobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In certain embodiments, the methods of the invention encompass the use of therapeutic antibodies known to be of use in treating B-cell malignancies; examples of such therapeutic antibodies include, but are not limited to, PANOREX™ (edrecolomab) which is a murine anti-17-IA cell surface antigen IgG2a antibody (Glaxo Wellcome/Centocor); BEC2 which is a murine anti-idiotype (GD3 epitope) IgG antibody (ImClone System); Erbitux® (cetuximab) which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ (rituximab) which is a chimeric anti-CD20 IgG1 antibody (IDEC Pharm/Genentech, Roche/Zettyaku); LYMPHOCIDE™ (epratuzumab) which is a humanized anti-CD22 IgG antibody (Immunomedics); ICM3 which is a humanized anti-ICAM3 antibody (ICOS Pharm); IDEC-114 which is a primatized anti-CD80 antibody (IDEC Pharm/Mitsubishi); ZEVALIN™ which is a radiolabelled murine anti-CD20 antibody (IDEC/Schering AG); IDEC-131 which is a humanized anti-CD40L antibody (IDEC/Eisai); IDEC-151 which is a primatized anti-CD4 antibody (IDEC); IDEC-152 which is a primatized anti-CD23 antibody (IDEC/Seikagaku); SMART anti-CD3 which is a humanized anti-CD3 IgG (Protein Design Lab); 5G1.1 which is a humanized anti-complement factor 5 (C5) antibody (Alexion Pharm); Humira® which is a human anti-TNF-α antibody (Abbott Laboratories); CDP870 which is a humanized anti-TNF-a Fab fragment (Celltech); IDEC-151 which is a primatized anti-CD4 IgG1 antibody (IDEC Pharm/SmithKline Beecham); MDX-CD4 which is a human anti-CD4 IgG antibody (Medarex/Eisai/Genmab); CDP571 which is a humanized anti-TNF-α IgG4 antibody (Celltech); LDP-02 which is a humanized anti-α4β7 antibody (LeukoSite/Genentech); OrthoClone OKT4A which is a humanized anti-CD4 IgG antibody (Ortho Biotech); ANTOVA™ which is a humanized anti-CD40L IgG antibody (Biogen); ANTEGREN™ which is a humanized anti-VLA-4 IgG antibody (Elan); and CAT-152 which is a human anti-TGF-β₂ antibody (Cambridge Ab Tech).

5.7 Subject Population

The invention provides methods for treating, preventing, and managing a plasma cell disorder, particularly systemic light-chain amyloidosis (AL) or multiple myeloma (MM), by administrating to a subject in need thereof a therapeutically or prophylactically effective amount of one or more compositions of the invention. In certain embodiments, the composition of the invention can be administered in combination with one or more other therapeutic agents. In other embodiments, the invention provides a method for diagnosis a plasma cell disorder in a subject comprising characterization of CD32B expression in the plasma cells of said subject. The subject is preferably a mammal such as a non-primate (e.g., cattle, swine, sheep, horses, cats, dogs, rodents, etc.) and a primate (e.g., monkey and a human). In a preferred embodiment, the subject is a human. In specific embodiments, the subject is an infant, a child, or an adult.

The methods and compositions of the invention comprise the administration or use of one or more compositions of the invention to subjects suffering from or expected to suffer from a plasma cell disorder, e.g., have a genetic predisposition for a plasma cell disorder or have suffered from a plasma cell disorder in the past or have been exposed to carcinogen or have been infected or previously exposed to cancer antigens. In a preferred embodiment, the subject or subject is predisposed or is suffering from malignant or benign plasma cell disorder. In specific embodiments, the subjects have pathological presentations of AL-amyloidosis or MM. In other embodiment, the subject suffers from one or more of the following: osteolytic lesions, recurrent bacterial infection, anemia, chronic interstitial nephritis, inflammation, monoclonal gammopathy, lupus nephritis, hemolytic uremic syndrome.

Such subjects may or may not have been previously treated for a plasma cell disorder. The methods and compositions of the invention may be used as a first line or second line treatment. Included in the invention is also the treatment of subjects currently undergoing therapies not comprising anti-CD32B antibodies, analogs, derivatives or fragments thereof to treat a plasma cell disorder. The invention also encompasses methods for administering one or more compositions of the invention to treat or ameliorate symptoms in refractory subjects. The invention also encompasses methods for administering one or more compositions of the invention to prevent the onset or recurrence of a plasma cell disorder or symptom thereof in subjects predisposed to having a plasma cell disorder.

In one embodiment, a subject may suffer from a plasma cell disorder is a subject who has or has had MM or MGUS. In other embodiments, the invention also provides methods of treatment of plasma cell disorders as an alternative to current therapies. In one embodiment, the current therapy has proven or may prove too toxic (i.e., results in unacceptable or unbearable side effects) for the subject (e.g., the use of dexamethasone). In another embodiment, the subject has proven refractory to the current therapy. In such embodiments, the invention provides administration of one or more compositions of the invention without any other plasma cell disorder therapies. In certain embodiments, one or more compositions of the invention can be administered to a subject in need thereof instead of another therapy to treat plasma cell disorders.

5.8 Prophylactic and Therapeutic Methods

The present invention encompasses therapies which involve administering an anti-CD32B antibody, analog, derivative or fragment thereof, preferably a humanized or human anti-CD32B antibody, to an animal, preferably a mammal, and most preferably a human, to prevent, treat, manage or ameliorate a plasma cell disorder, or one or more symptoms thereof. These therapies are an enhancement over current therapies. In certain cases, subjects who are refractory to current therapies can be treated with the methods of the invention. In some embodiments, therapy by administration of one or more antibodies of the invention is combined with administration of one or more therapies such as, but not limited to, chemotherapies, radiation therapies, hormonal therapies, and/or biological therapies/immunotherapies.

Prophylactic and therapeutic compounds of the invention include, but are not limited to, proteinaceous molecules, including, but not limited to, peptides, polypeptides, proteins, including post-translationally modified proteins, antibodies, etc.; small molecules (less than 1000 Daltons), inorganic or organic compounds; nucleic acid molecules including, but not limited to, double-stranded or single-stranded DNA, double-stranded or single-stranded RNA, as well as triple helix nucleic acid molecules. Prophylactic and therapeutic compounds can be derived from any known organism (including, but not limited to, animals, plants, bacteria, fungi, and protista, or viruses) or from a library of synthetic molecules.

Antibodies may be provided in pharmaceutically acceptable compositions as known in the art or as described herein.

Antibodies used in the methods of the present invention that function as a prophylactic and/or therapeutic agent of a disease, disorder, or infection can be administered to an animal, preferably, a mammal and most preferably, a human, to treat, prevent or ameliorate a plasma cell disorder, or one or more symptoms thereof. Antibodies used in the methods of the invention can be administered in combination with one or more other prophylactic and/or therapeutic agents useful in the treatment, prevention or management of a plasma cell disorder, or one or more symptoms thereof. In certain embodiments, one or more antibodies used in the methods of the invention are administered to a mammal, preferably, a human, concurrently with one or more other therapeutic agents useful for the treatment of cancer or plasma cell malignancy. The term “concurrently” is not limited to the administration of prophylactic or therapeutic agents at exactly the same time, but rather it is meant that antibodies used in the methods of the invention and the other agent are administered to a subject in a sequence and within a time interval such that the antibodies used in the methods of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same subject visit.

The dosage amounts and frequencies of administration provided herein are encompassed by the terms therapeutically effective and prophylactically effective. The dosage and frequency further will typically vary according to factors specific for each subject depending on the specific therapeutic or prophylactic agents administered, the severity and type of cancer, the route of administration, as well as age, body weight, response, and the past medical history of the subject. Suitable regimens can be selected by one skilled in the art by considering such factors and by following, for example, dosages reported in the literature and recommended in the Physician's Desk Reference (56^{th} ed., 2002).

5.9 Compositions and Methods of Administering

The invention provides methods and pharmaceutical compositions comprising anti-CD32B antibodies, analogs, derivatives, or fragments thereof. The invention also provides methods of treatment, prophylaxis, and amelioration of a plasma cell disorder or one or more symptoms thereof by administering to a subject an effective amount of a fusion protein or a conjugated molecule comprising a an anti-CD32B antibody, analog, derivative or fragment thereof or a pharmaceutical composition comprising a fusion protein or conjugated molecules comprising an anti-CD32B antibody analog, derivative or fragment thereof. In a preferred aspect, an antibody or fusion protein or conjugated molecule, is substantially purified (i.e., substantially free from substances that limit its effect or produce undesired side-effects). In a specific embodiment, the subject is an animal, preferably a mammal such as non-primate (e.g., cows, pigs, horses, cats, dogs, rats etc.) and a primate (e.g., monkey such as, a cynomolgous monkey and a human). In a preferred embodiment, the subject is a human.

Various delivery systems are known and can be used to administer a composition comprising humanized antibodies of the invention, e.g., encapsulation in liposomes, microparticles, microcapsules, recombinant cells capable of expressing the antibody or fusion protein, receptor-mediated endocytosis (see, e.g., Wu and Wu, 1987, J. Biol. Chem. 262:4429-4432), construction of a nucleic acid as part of a retroviral or other vector, etc.

In some embodiments, the antibodies used in the methods of the invention are formulated in liposomes for targeted delivery of the antibodies of the invention. Liposomes are vesicles comprised of concentrically ordered phospholipid bilayers which encapsulate an aqueous phase. Liposomes typically comprise various types of lipids, phospholipids, and/or surfactants. The components of liposomes are arranged in a bilayer configuration, similar to the lipid arrangement of biological membranes. Liposomes are particularly preferred delivery vehicles due, in part, to their biocompatibility, low immunogenicity, and low toxicity. Methods for preparation of liposomes are known in the art and are encompassed within the invention, see, e.g., Epstein et al., 1985, Proc. Natl. Acad. Sci. USA, 82: 3688; Hwang et al., 1980 Proc. Natl. Acad. Sci. USA, 77: 4030-4; U.S. Pat. Nos. 4,485,045 and 4,544,545; all of which are incorporated herein by reference in their entirety.

Methods of preparing liposomes with a prolonged serum half-life, i.e., enhanced circulation time, are disclosed in U.S. Pat. No. 5,013,556. Preferred liposomes are not rapidly cleared from circulation, i.e., are not taken up into the mononuclear phagocyte system (MPS). The invention encompasses sterically stabilized liposomes which are prepared using common methods known to one skilled in the art. Although not intending to be bound by a particular mechanism of action, sterically stabilized liposomes contain lipid components with bulky and highly flexible hydrophilic moieties, which reduces the unwanted reaction of liposomes with serum proteins, reduces opsonization with serum components and reduces recognition by MPS. Sterically stabilized liposomes are preferably prepared using polyethylene glycol. For preparation of liposomes and sterically stabilized liposome, see, e.g., Bendas et al., 2001 BioDrugs, 15(4): 215-224; Allen et al., 1987 FEBS Lett. 223: 42-6; Klibanov et al., 1990 FEBS Lett., 268: 235-7; Blum et al., 1990, Biochim. Biophys. Acta., 1029: 91-7; Torchilin et al., 1996, J. Liposome Res. 6: 99-116; Litzinger et al., 1994, Biochim. Biophys. Acta, 1190: 99-107; Maruyama et al., 1991, Chem. Pharm. Bull., 39: 1620-2; Klibanov et al., 1991, Biochim Biophys Acta, 1062; 142-8; Allen et al., 1994, Adv. Drug Deliv. Rev, 13: 285-309; all of which are incorporated herein by reference in their entirety. The invention also encompasses liposomes that are adapted for specific organ targeting, see, e.g., U.S. Pat. No. 4,544,545. Particularly useful liposomes for use in the compositions and methods of the invention can be generated by reverse phase evaporation method with a lipid composition comprising phosphatidylcholine, cholesterol, and PEG derivatized phosphatidylethanolamine (PEG-PE). Liposomes are extruded through filters of defined pore size to yield liposomes with the desired diameter. In some embodiments, a fragment of an antibody of the invention, e.g., F(ab′), may be conjugated to the liposomes using previously described methods, see, e.g., Martin et al., 1982, J. Biol. Chem. 257: 286-288, which is incorporated herein by reference in its entirety.

The antibodies used in the methods of the invention may also be formulated as immunoliposomes. Immunoliposomes refer to a liposomal composition, wherein an antibody of the invention or a fragment thereof is linked, covalently or non-covalently to the liposomal surface. The chemistry of linking an antibody to the liposomal surface is known in the art and encompassed within the invention, see, e.g., Allen et al., 1995, Stealth Liposomes, Boca Rotan: CRC Press, 233-44; Hansen et al., 1995, Biochim. Biophys. Acta, 1239: 133-44; which are incorporated herein by reference in their entirety. In most preferred embodiments, immunoliposomes for use in the methods and compositions of the invention are further sterically stabilized. Preferably, the antibodies used in the methods of the invention are linked covalently or non-covalently to a hydrophobic anchor, which is stably rooted in the lipid bilayer of the liposome. Examples of hydrophobic anchors include, but are not limited to, phospholipids, e.g., phosphatidylethanolamine (PE), phosphatidylinositol (PI). To achieve a covalent linkage between an antibody and a hydrophobic anchor, any of the known biochemical strategies in the art may be used, see, e.g., J. Thomas August, ed., 1997, Gene Therapy: Advances in Pharmacology, Volume 40, Academic Press, San Diego, Calif., p. 399-435, which is incorporated herein by reference in its entirety. For example, a functional group on an antibody molecule may react with an active group on a liposome associated hydrophobic anchor, e.g., an amino group of a lysine side chain on an antibody may be coupled to liposome associated N-glutaryl-phosphatidylethanolamine activated with water-soluble carbodiimide; or a thiol group of a reduced antibody can be coupled to liposomes via thiol reactive anchors, such as pyridylthiopropionyl-phosphatidylethanolamine. See, e.g., Dietrich et al., 1996, Biochemistry, 35: 1100-1105; Loughrey et al., 1987, Biochim. Biophys. Acta, 901: 157-160; Martin et al., 1982, J. Biol. Chem. 257: 286-288; Martin et al., 1981, Biochemistry, 20: 4429-38; all of which are incorporated herein by reference in their entirety. Although not intending to be bound by a particular mechanism of action, immunoliposomal formulations comprising an antibody of the invention are particularly effective as therapeutic agents, since they deliver the antibody to the cytoplasm of the target cell, i.e., the cell comprising the Fc□RIIB receptor to which the antibody binds. The immunoliposomes preferably have an increased half-life in blood, specifically target cells, and can be internalized into the cytoplasm of the target cells thereby avoiding loss of the therapeutic agent or degradation by the endolysosomal pathway.

The invention encompasses immunoliposomes comprising a humanized antibody of the invention or a fragment thereof. In some embodiments, the immunoliposomes further comprise one or more additional therapeutic agents, such as those disclosed herein.

The immunoliposomal compositions used in the methods of the invention comprise one or more vesicle forming lipids, an antibody of the invention or a fragment or derivative thereof, and, optionally, a hydrophilic polymer. A vesicle forming lipid is preferably a lipid with two hydrocarbon chains, such as acyl chains and a polar head group. Examples of vesicle forming lipids include phospholipids, e.g., phosphatidylcholine, phosphatidylethanolamine, phosphatidic acid, phosphatidylinositol, sphingomyelin, and glycolipids, e.g., cerebrosides, gangliosides. Additional lipids useful in the formulations of the invention are known to one skilled in the art and encompassed within the invention. In some embodiments, the immunoliposomal compositions further comprise a hydrophilic polymer, e.g., polyethylene glycol, and ganglioside GM1, which increases the serum half life of the liposome. Methods of conjugating hydrophilic polymers to liposomes are well known in the art and encompassed within the invention. For a review of immunoliposomes and methods of preparing them, see, e.g., PCT International Publication No. WO 97/38731, Vingerhoeads et al., 1994, Immunomethods, 4: 259-72; Maruyama, 2000, Biol. Pharm. Bull. 23(7): 791-799; Abra et al., 2002, Journal of Liposome Research, 12 (1&2): 1-3; Park, 2002, Bioscience Reports, 22(2): 267-281; Bendas et al., 2001 BioDrugs, 14(4): 215-224, J. Thomas August, ed., 1997, Gene Therapy: Advances in Pharmacology, Volume 40, Academic Press, San Diego, Calif., p. 399-435, all of which are incorporated herein by reference in their entireties.

Methods of administering a compositions of the invention include, but are not limited to, parenteral administration (e.g., intradermal, intramuscular, intraperitoneal, intravenous and subcutaneous), epidural, and mucosal (e.g., intranasal and oral routes). In a specific embodiment, the compositions of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,20; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that the anti-CD32B antibodies, analogs, derivatives, or fragments thereof are packaged in a hermetically sealed container, such as an ampoule or cachet, indicating the quantity of antibody. In one embodiment, the anti-CD32B-antibody, analog, derivative, or fragment thereof is supplied as a dry sterilized lyophilized powder or water free concentrate in a hermetically sealed container and can be reconstituted, e.g., with water or saline to the appropriate concentration for administration to a subject. Preferably, the anti-CD32B-antibody, analog, derivative, or fragment thereof is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized compositions of the invention should be stored at between 2 and 8° C. in their original container and, for the case of antibodies, should be administered within 12 hours, preferably within 6 hours, within 5 hours, within 3 hours, or within 1 hour after being reconstituted. In an alternative embodiment, the anti-CD32B-antibody, analog, derivative, or fragment thereof is supplied in liquid form in a hermetically sealed container indicating the quantity and concentration of the antagonist, antibody, fusion protein, or conjugated molecule. Preferably, the liquid form of the composition of the invention is supplied in a hermetically sealed container at least 1 mg/ml, more preferably at least 2.5 mg/ml, at least 5 mg/ml, at least 8 mg/ml, at least 10 mg/ml, at least 15 mg/kg, at least 25 mg/ml, at least 50 mg/ml, at least 100 mg/ml, at least 150 mg/ml, at least 200 mg/ml of the anti-CD32B-antibody, analog, derivative, or fragment thereof.

The amount of the compositions used in the methods of the invention which will be effective in the treatment, prevention or amelioration of a plasma cell disorder, or one or more symptoms thereof, can be determined by standard clinical techniques. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

For antibodies encompassed by the invention, the dosage administered to a subject is typically 0.0001 mg/kg to 100 mg/kg of the subject's body weight. Preferably, the dosage administered to a subject is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the subject's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In one embodiment, the dosage of the antibodies used in the methods of the invention administered to a subject are 0.01 mg to 1000 mg/day, when used as single agent therapy. In another embodiment the antibodies used in the methods of the invention are used in combination with other therapeutic compositions and the dosage administered to a subject are lower than when said antibodies are used as a single agent therapy.

In a specific embodiment, it may be desirable to administer the pharmaceutical compositions used in the methods of the invention locally to the area in need of treatment; this may be achieved by, for example, and not by way of limitation, local infusion, by injection, or by means of an implant, said implant being of a porous, non-porous, or gelatinous material, including membranes, such as silastic membranes, or fibers. Preferably, when administering an antibody used in the methods of the invention, care must be taken to use materials to which the antibody or the fusion protein does not absorb.

In another embodiment, the compositions can be delivered in a vesicle, in particular a liposome (See Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 3 17-327; see generally ibid.).

In yet another embodiment, the compositions can be delivered in a controlled release or sustained release system. Any technique known to one of skill in the art can be used to produce sustained release formulations comprising one or more antibodies of the invention. See, e.g., U.S. Pat. No. 4,526,938; PCT publication WO 91/05548; PCT publication WO 96/20698; Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397; Cleek et al., 1997, “Biodegradable Polymeric Carriers for a bFGF Antibody for Cardiovascular Application,” Pro. Int'l. Symp. Control. Rel. Bioact. Mater. 24:853-854; and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in its entirety. In one embodiment, a pump may be used in a controlled release system (See Langer, supra; Sefton, 1987, CRC Crit. Ref. Biomed. Eng. 14:20; Buchwald et al., 1980, Surgery 88:507; and Saudek et al., 1989, N. Engl. J. Med. 321:574). In another embodiment, polymeric materials can be used to achieve controlled release of antibodies (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; See also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycosides) (PLGA), and polyorthoesters. In yet another embodiment, a controlled release system can be placed in proximity of the therapeutic target (e.g., the lungs), thus requiring only a fraction of the systemic dose (see, e.g., Goodson, in Medical Applications of Controlled Release, supra, vol. 2, pp. 115-138 (1984)). In another embodiment, polymeric compositions useful as controlled release implants are used according to Dunn et al. (See U.S. Pat. No. 5,945,155). This particular method is based upon the therapeutic effect of the in situ controlled release of the bioactive material from the polymer system. The implantation can generally occur anywhere within the body of the subject in need of therapeutic treatment. In another embodiment, a non-polymeric sustained delivery system is used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

In a specific embodiment where the composition of the invention is a nucleic acid encoding an antibody, the nucleic acid can be administered in vivo to promote expression of its encoded antibody, by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (See e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

For antibodies, the therapeutically or prophylactically effective dosage administered to a subject is typically 0.1 mg/kg to 200 mg/kg of the subject's body weight. Preferably, the dosage administered to a subject is between 0.1 mg/kg and 20 mg/kg of the subject's body weight and more preferably the dosage administered to a subject is between 1 mg/kg to 10 mg/kg of the subject's body weight. The dosage and frequency of administration of antibodies of the invention may be reduced also by enhancing uptake and tissue penetration (e.g., into the lung) of the antibodies or fusion proteins by modifications such as, for example, lipidation.

Treatment of a subject with a therapeutically or prophylactically effective amount of an anti-CD32B-antibody, analog, derivative, or fragment thereof can include a single treatment or, preferably, can include a series of treatments. In a preferred example, a subject is treated with an anti-CD32B-antibody, analog, derivative, or fragment thereof in the range of between about 0.1 to 30 mg/kg body weight, one time per week for between about 1 to 10 weeks, preferably between 2 to 8 weeks, more preferably between about 3 to 7 weeks, and even more preferably for about 4, 5, or 6 weeks. In other embodiments, the pharmaceutical compositions used in the methods of the invention are administered once a day, twice a day, or three times a day. In other embodiments, the pharmaceutical compositions are administered once a week, twice a week, once every two weeks, once a month, once every six weeks, once every two months, twice a year or once per year. It will also be appreciated that the effective dosage of the anti-CD32B-antibody, analog, derivative, or fragment thereof used for treatment may increase or decrease over the course of a particular treatment.

5.9.1 Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or subject) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of humanized antibodies of the invention and a pharmaceutically acceptable carrier.

In one particular embodiment, the pharmaceutical composition comprises of a therapeutically effective amount of a humanized antibody or a fragment thereof that binds FcγRIIB with a greater affinity than said antibody or a fragment thereof binds FcγRIIA, a cytotoxic antibody that specifically binds a cancer antigen, and a pharmaceutically acceptable carrier. In another embodiment, said pharmaceutical composition further comprises one or more anti-cancer agents.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or cachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

5.10 Characterization and Demonstration of Therapeutic Utility

Several aspects of the pharmaceutical compositions or prophylactic or therapeutic agents of the invention are preferably tested in vitro, e.g., in a cell culture system, and then in vivo, e.g., in an animal model organism, such as a rodent animal model system, for the desired therapeutic activity prior to use in humans. For example, assays which can be used to determine whether administration of a specific pharmaceutical composition is indicated, include cell culture assays in which a subject tissue sample is grown in culture, and exposed to or otherwise contacted with a pharmaceutical composition, and the effect of such composition upon the tissue sample is observed, e.g., inhibition of or decrease in growth and/or colony formation in soft agar or tubular network formation in three-dimensional basement membrane or extracellular matrix preparation. The tissue sample can be obtained by biopsy from the subject. This test allows the identification of the therapeutically most effective prophylactic or therapeutic molecule(s) for each individual subject. Alternatively, instead of culturing cells from a subject, therapeutic agents and methods may be screened using cells of a tumor or malignant cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, decreased growth and/or colony formation in soft agar or tubular network formation in three-dimensional basement membrane or extracellular matrix preparation, etc. Additional assays include raft association, CDC, ADCC and apoptosis assays as known in the art and described in the Examples.

Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, combinations of prophylactic and/or therapeutic agents are tested in a mouse model system. Such model systems are widely used and well-known to the skilled artisan. Prophylactic and/or therapeutic agents can be administered repeatedly. Several aspects of the procedure may vary such as the temporal regime of administering the prophylactic and/or therapeutic agents, and whether such agents are administered separately or as an admixture.

Preferred animal models for use in the methods of the invention are, for example, transgenic mice expressing FcγR on mouse effector cells, e.g., any mouse model described in U.S. Pat. No. 5,877,396 (which is incorporated herein by reference in its entirety). Transgenic mice for use in the methods of the invention include, but are not limited to, mice carrying human FcγRIIIA, mice carrying human FcγRIIA, mice carrying human FcγRIIB and human FcγRIIIA, mice carrying human FcγRIIB and human FcγRIIA.

Once the prophylactic and/or therapeutic agents of the invention have been tested in an animal model they can be tested in clinical trials to establish their efficacy. Establishing clinical trials will be done in accordance with common methodologies known to one skilled in the art, and the optimal dosages and routes of administration as well as toxicity profiles of the compositions of the invention can be established using routine experimentation.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer such as the SCID mouse model or transgenic mice or nude mice with human xenografts, animal models, such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

The protocols and compositions of the invention are preferably tested in vitro, and then in vivo, for the desired therapeutic or prophylactic activity, prior to use in humans. Therapeutic agents and methods may be screened using cells of a tumor or malignant cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, decreased growth and/or colony formation in soft agar or tubular network formation in three-dimensional basement membrane or extracellular matrix preparation, etc.

Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

Further, any assays known to those skilled in the art can be used to evaluate the prophylactic and/or therapeutic utility of the combinatorial therapies disclosed herein for treatment or prevention of cancer, inflammatory disorder, or autoimmune disease.

5.11 Preparation of CD32B-Specific Antibodies

The invention encompasses nucleotide sequences that encode the CDR-grafted heavy and light chains, cloning and expression vectors containing the nucleotide sequences, host cells transformed with the nucleotide sequences, and methods for the production of the CDR-grafted chains and antibody molecules comprising the nucleotide sequences in the transformed host cells. In specific embodiments, the invention encompasses any of the nucleotide sequences of SEQ ID NO:31, SEQ ID NO:32, SEQ ID NO:33, SEQ ID NO:34, SEQ ID NO:35 or SEQ ID NO:36.

The invention encompasses donor amino acid sequences, which encode antibodies that bind Fc□RIIB with a greater affinity that Fc□RIIA. In a specific embodiment, the donor amino acid sequence encodes for the monoclonal antibody produced from clone 2B6 or 3H7, with ATCC accession numbers PTA-4591 and PTA-4592, respectively, or other monoclonal antibodies produced by immunization methods of the invention as disclosed in U.S. Patent Application Publication Nos. US 2005/02157667 and US 2004/0185045, both of which are incorporated herein by reference in their entireties In another specific embodiment, the donor amino acid sequence encodes for the monoclonal antibody produced from clone 1D5, 2E1, 2H9, 2D11, or 1F2, with ATCC accession numbers PTA-5958, PTA-5961, PTA-5962, PTA-5960, and PTA-5959, respectively, or other monoclonal antibodies produced by immunization methods of the invention as disclosed in U.S. Provisional Application No. 60/403,366, filed on Aug. 14, 2002 and U.S. Patent Application Publication No. 2004/0185045, both of which are incorporated herein by reference in their entireties. The invention also encompass polynucleotides that encode for donor amino acid sequences that hybridize under various stringency, e.g., high stringency, intermediate or low stringency conditions, to polynucleotides that encode for the monoclonal antibody produced from clone 2B6, 3H7, 1D5, 2E1, 2H9, 2D11, or 1F2, with ATCC accession numbers PTA-4591, PTA-4592, PTA-5958, PTA-5961, PTA-5962, PTA-5960, and PTA-5959, respectively, or other monoclonal antibodies produced by immunization methods of the invention as disclosed in U.S. Provisional Application No. 60/403,366, filed on Aug. 14, 2002 and U.S. Patent Application Publication No. 2004/0185045. The hybridization can be performed under various conditions of stringency. By way of example and not limitation, procedures using conditions of low stringency are as follows (see also Shilo and Weinberg, 1981, Proc. Natl. Acad. Sci. U.S.A. 78, 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5−20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations). By way of example and not limitation, procedures using conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5−20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art. Selection of appropriate conditions for such stringencies is well known in the art (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, © 1987-1997, Current Protocols, © 1994-1997 John Wiley and Sons, Inc.; see especially, Dyson, 1991, “Immobilization of nucleic acids and hybridization analysis,” In: Essential Molecular Biology: A Practical Approach, Vol. 2, T. A. Brown, ed., pp. 111-156, IRL Press at Oxford University Press, Oxford, UK). The polynucleotides may be obtained, and the nucleotide sequence of the polynucleotides determined, by any method known in the art.

DNA sequences which encode the acceptor amino acid sequences may be obtained by any method known to one skilled in the art. For example, DNA sequences coding for preferred human acceptor framework sequences include but are not limited to FR segments from the human germline VH segment VH1-8 and JH6 and the human germline VL segment VK-A26 and JK4.

In a specific embodiment, one or more of the CDRs are inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to FcγRIIB with greater affinity than said antibody binds FcγRIIA. Preferably, as discussed supra, one or more amino acid substitutions may be made within the framework regions, and, preferably, the amino acid substitutions improve binding of the antibodies of the invention to FcγRIIB.

In another embodiment, human libraries or any other libraries available in the art, can be screened by standard techniques known in the art, to clone the nucleic acids encoding the antibodies of the invention.

The antibodies used in the methods of the present invention may be produced by any method known in the art useful for the production of polypeptides, e.g., in vitro synthesis, recombinant DNA production, and the like. Preferably, the humanized antibodies are produced by recombinant DNA technology. The humanized Fc□RIIB specific antibodies of the invention may be produced using recombinant immunoglobulin expression technology. The recombinant production of immunoglobulin molecules, including humanized antibodies are described in U.S. Pat. No. 4,816,397 (Boss et al.), U.S. Pat. Nos. 6,331,415 and 4,816,567 (both to Cabilly et al.), U.K. patent GB 2,188,638 (Winter et al.), and U.K. patent GB 2,209,757; all of which are incorporated herein by reference in their entireties. Techniques for the recombinant expression of immunoglobulins, including humanized immunoglobulins, can also be found, in Goeddel et al., Gene Expression Technology Methods in Enzymology Vol. 185 Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993).

The general methods for construction of the vectors of the invention, transfection of cells to produce the host cell of the invention, culture of cells to produce the antibody of the invention are all conventional molecular biology methods. Likewise, once produced, the recombinant humanized antibodies of the invention may be purified by standard procedures of the art, including cross-flow filtration, ammonium sulfate precipitation, affinity column chromatography, gel electrophoresis and the like.

The CD32B-specific antibodies used in the methods of the present invention may be used in conjunction with, or attached to, other antibodies (or parts thereof) such as human or humanized monoclonal antibodies. These other antibodies may be reactive with other markers (epitopes) characteristic for the disease against which the antibodies of the invention are directed or may have different specificities chosen, for example, to recruit molecules or cells of the human immune system to the diseased cells. The antibodies used in the methods of the invention (or parts thereof) may be administered with such antibodies (or parts thereof) as separately administered compositions or as a single composition with the two agents linked by conventional chemical or by molecular biological methods. Additionally the diagnostic and therapeutic value of the antibodies used in the methods of the invention may be augmented by labeling the humanized antibodies with labels that produce a detectable signal (either in vitro or in vivo) or with a label having a therapeutic property. Some labels, e.g., radionuclides, may produce a detectable signal and have a therapeutic property. Examples of radionuclide labels include, but are not limited to, ¹²⁵I, ¹³¹I, and ¹⁴C. Examples of other detectable labels include a fluorescent chromophore such as fluorescein, phycobiliprotein or tetraethyl rhodamine for fluorescence microscopy, an enzyme which produces a fluorescent or colored product for detection by fluorescence, absorbance, visible color or agglutination, which produces an electron dense product for demonstration by electron microscopy; or an electron dense molecule such as ferritin, peroxidase or gold beads for direct or indirect electron microscopic visualization. Labels having therapeutic properties include drugs for the treatment of cancer, such as methotrexate and the like.

The subject invention provide numerous antibodies specific for the FcγRIIB based on the discovery that the CDR regions of the murine monoclonal antibody could be spliced into a human acceptor framework so as to produce a humanized recombinant antibody specific for the FcγRIIB. Preferred humanized FcγRIIB specific antibodies contain an additional change in the framework region (or in other regions) to increasing binding for FcγRIIB. Particularly preferred embodiments of the invention are the exemplified humanized antibody molecules that have superior binding properties for FcγRIIB.

The invention encompasses standard recombinant DNA methods for preparing DNA sequences which code for the CDR-grafted antibodies of the invention. DNA sequences may be synthesized completely or in part using oligonucleotide synthesis techniques. Methods for oligonucleotide directed synthesis are well known in the art. The invention further encompasses site-directed mutagenesis methods such as those known in the art.

Any suitable host cell/vector system may be used for expression of the DNA sequences coding for the CDR-grafted heavy and light chains. Bacterial, e.g., E. coli, and other microbial systems may be used, in particular for expression of antibody fragments such as Fab and (Fab′)₂ fragments, and especially FV fragments and single chain antibody fragments, e.g., single chain FVs. Eucaryotic systems, e.g., mammalian host cell expression systems, may be used for production of larger CDR-grafted antibody products, including complete antibody molecules. Suitable mammalian host cells include CHO cells and myeloma or hybridoma cell lines. Other cell lines that may be used include, but are not limited to, CHO-K1, NSO, and PER.C6 (Crucell, Leiden, Netherlands).

The donor murine antibodies may be produced using any method known in the art, including those disclosed in U.S. application Ser. No. 10/643,857 filed Aug. 14, 2003; and U.S. Provisional Application No. 60/403,266 filed on Aug. 14, 2002; both of which are incorporated herein by reference in their entireties.

Antibody fragments which recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the complete light chain, and the variable region, the CH1 region and at least a portion of the hinge region of the heavy chain.

As known in the art, phage display technology can be used to increase the affinity of an antibody of the invention for FcγRIIB. This technique would be useful in obtaining high affinity antibodies that could be used in the combinatorial methods of the invention. This technology, referred to as affinity maturation, employs mutagenesis or CDR walking and re-selection using FcγRIIB or an antigenic fragment thereof to identify antibodies that bind with higher affinity to the antigen when compared with the initial or parental antibody (See, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased binding affinity for the antigen can be screened by contacting the immobilized mutants with labeled antigen. Any screening method known in the art can be used to identify mutant antibodies with increased avidity to the antigen (e.g., ELISA) (See Wu et al., 1998, Proc Natl. Acad. Sci. USA 95:6037; Yelton et al., 1995, J. Immunology 155:1994). CDR walking which randomizes the light chain is also possible (See Schier et al., 1996, J. Mol. Bio. 263:551).

5.12 Kits

The invention provides a pharmaceutical pack or kit comprising one or more containers filled with a CD32B antagonist used in the methods of the invention. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more CD32B antagonists used in the methods of the invention. In another embodiment, a kit further comprises one or more other prophylactic or therapeutic agents useful for the treatment of cancer, in one or more containers. In another embodiment, a kit further comprises one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

### 6. EXAMPLES

### 6.1 Gene and Cell Surface Expression of CD32B in Plasma Cell Samples from Subjects with Plasma Cell Disorders

The expression of CD32B in plasma cells of subjects diagnosed with light-chain amyloidosis (AL), multiple myeloma (MM), or plasma cell leukemia (PCL) was examined at both the transcriptional and translational level by assessing CD32B mRNA and cell surface protein expression, respectively. CD32B mRNA and protein was found to be expressed at a high level in all of the AL samples tested and in a majority of the MM samples. Plasma cells from subjects with PCL and control MM cell lines were found to express CD32B protein at a lower level. Cytogenetic examination of MM samples that did not highly express CD32B showed chromosomal abnormalities associated with unfavorable prognosis. Accordingly, the level of CD32B surface protein expression on plasma cells has prognostic significance as it is inversely proportional to biologic aggressivity.

Materials and Methods

Plasma Cell Isolation. Plasma cells were isolated from mononuclear fractions of bone marrow aspirates (AL, MM) or blood samples (PCL) by fluorescent activated cell sorting (FACS) or by immunomagnetic separation.

For FACS, mononuclear cells were incubated with PE-conjugated anti-CD138 murine monoclonal antibody, B-B4 (Miltenyi Biotech, Auburn, Calif.) to stain the plasma cell population. The mononuclear cells were also incubated in 4′-6-Diamidino-2-phenylindole (DAPI; Invitrogen, Carlsbad, Calif.) to stain non-viable cells. CD138 positive/DAPI negative cells were selected by FACS and used for gene-expression profile (GEP) analysis.

For immunomagnetic separation, CD138 positive cells were separated from mononuclear cells using B-B4 antibody coated microbeads and the MiniMACS separator according to the manufacturer's directions (Miltenyi Biotech, Aurburn, Calif.). Selected cells were subject to a second selection to ensure purity. Immunomagnetically selected plasma cells were subsequently used for RT-PCR analysis, immunofluorescent microscopy, and flow cytometry.

Flow cytometry: Isolated, CD138 positive plasma cells were incubated with PE conjugated anti-CD138 antibody (clone MI15, BD Biosciences, San Jose, Calif. or clone B-B4, Miltenyi Biotech, Aurburn, Calif.) and Alexa Fluor 488-conjugated anti-CD32B antibody (clone 2B6, MacroGenics, Rockville, Md.) for 30 min at 4° C. Control samples were incubated with non-reactive isotype matched antibodies. Samples were washed, fixed with 4% paraformaldehyde, and analyzed using a Cytomics FC 500 System (Beckman Coulter, Miami, Fla.).

Reverse-Transcriptase Polymerase Chain Reaction (RT-PCR): RNA was isolated from CD138 positive cells using an RNEasy kit according the manufacturer's directions (Qiagen, Valencia, Calif.). Single stranded cDNA was synthesized by reverse transcriptase. The CD32B message was amplified from cDNA using forward primer 5′-CCTCACCTGGAGTTCCAGGAGGGAG-3′ (SEQ ID NO:37) and reverse primer 5′-AACTTTGTCAGCCTCATCAGG-3′ (SEQ ID NO:38). The primers amplify both CD32B isoforms, CD32B1 and CD32B2. The amplicon for CD32B1 is 441 base pairs (nt 504-944 of the cDNA), and the amplicon of CD32B is 364 base pairs (nt 457-820 of the cDNA). Amplification of the of the GAPDH message from the prepared cDNA was used as a control (forward primer: 5′-TTCGACAGTCAGCC GCATCTTCTT-3′ (SEQ ID NO:39; reverse: 5′-GCCCAATACGACCAAATCCGTTGA-3′ (SEQ ID NO:40; amplicon 100 bp). The conditions for amplification were pre-denaturation 94° C., 2 minutes; denaturation 94° C., 40s; annealing at 58° C., 40s; extension at 72° C., 60s; 40 cycles.

Gene Expression Profiles: RNA was extracted from FACS-sorted CD138+/DAPI-plasma cells as described for RT-PCR, supra. cRNA was made and processed, and Affymetrix U133 PLUS 2.0 arrays performed according to manufacturer's instructions. Data were vetted based on plasma cell lineage gene expression; samples contaminated with monocytes were not used. Expression data was normalized and expression levels identified for Affymetrix U133 PLUS 2.0 oligomers for members of the Fc-receptor family: 210889 (CD32B), 293561 (CD32A), 204007 (CD16) and 216951 (CD64).

Results:

All subjects were either newly diagnosed cases of AL or MM, or had relapsed disease. Plasma cells from subjects with PCL served as a control representing a highly aggressive species of plasma cell disorder. CD138 selected samples by FACS or immunomagnetic separation were greater than 95% pure by immunofluorescent microscopy.

CD32B gene expression: CD32B mRNA expression was found in all GEP tested AL samples and in the public ally available GEP data for MM (Affymetrix HuGeneFL, http://lambertlab.uams.edu/software/). Comparison of the normalized expression levels of Fc-receptor family member messages revealed that expression of the message for CD32B was significantly greater than that for CD32A, CD16, or CD64 by paired t-test (p<0.01). Similarly, analysis of the public GEP data for MM of revealed that CD32B message expression was higher than that that of other Fc-receptor genes.

CD32B mRNA was also detected in subject samples of CD138 positive cells by RT-PCR. Messages for both isoforms of CD32B, CD32B1 and CD32B2, were found in plasma cell isolates regardless of disease (i.e., AL, MM, or PCL) or disease state (i.e., newly diagnosed or relapsed), with the message for CD32B1 consistently expressed at a qualitatively higher level than that of CD32B2. In contrast, RT-PCR analysis of the MM cell line RPMI 8226 failed to detect and CD32B message.

CD32B protein expression: The disparity between CD32B message expression in primary MM samples and a model MM cell line may reflect the inherent variation in CD32B expression of clonal MM cells. Neither GEP nor RT-PCR provide information with respect to variation within a cell sample, producing only an “averaged” expression or expression profile of the cells within the sample. Thus, flow cytometry was used to investigate whether CD138 positive cell samples from AL, MM, and PCL subjects exhibited variation in CD32B surface protein expression.

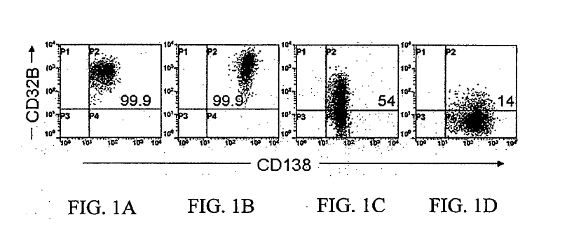

Flow cytometric analysis showed intense cell surface staining for CD32B on greater than 98% of plasma cells, i.e., CD138 positive cells, for all samples isolated from AL subjects (FIG. 1A). In contrast, the CD32B expression of CD138 positive cells isolated from MM subjects showed greater variability (median 94%, range 1-100%; n=23; FIGS. 1B-1C). Comparison of the percentage of CD32B positive cells in the MM samples revealed that the MM subjects fell into two categories: those whose plasma cells exhibited high CD32B expression (median 96.5% CD32B+, range 90-100%; FIG. 1B), and those whose plasma cells exhibited significantly lower expression (median 42% CD32B+, range 1-85%; FIG. 1C). Cytogenetic and fluorescent in-situ hybridization (FISH) analysis of the plasma cells from the high expression subjects showed normal or hyperdiploid karyotype, while analysis of the plasma cells of lower expressing subjects showed abnormalities associated with unfavorable prognosis (hypodiploid complex cytogenetics, 13q deletions, and translocation t(4:14)(p16; q32)). A statistical comparison by two tailed Mann-Whitney had a p-value of 0.0001, indicating a highly statistical difference.

Flow cytometry also revealed the absence of CD32B surface protein in MM cell lines (PRMI 8226, OPM-2, SKMM1, and SKMM2) and low CD32B expression on plasma cells from subjects with plasma cell leukemia (PCL) (median 28%; FIG. 1D). Accordingly, it was concluded that a reduction in CD32B expression was correlated with unfavorable prognosis/aggressivity.

GEP analysis of CD138+/CD32B− plasma cells from MM subjects: The apparent correlation between CD32B expression and clinical prognosis lead to the study of CD138 positive/CD32B negative plasma cells isolated from MM subjects. Cytogenetic analysis of the CD138+/CD32B− fraction revealed an overexpression of genes on chromosome 1, including genes for cancer/testis antigens and others commonly associated with biologic aggressivity in MM (Carrasco et al., 2006, Cancer Cell 9:313-325).

6.2 In Vivo Activity of FCγRIIB Antibodies in Xenograft Murine Models with Human Multiple Myeloma Derived Cells

The ability to prevent tumors in a mouse model of a plasma cell disorder, e.g., multiple myeloma is an important criterion to determine the potential for an antibody to proceed into clinical studies. Murine models using primary samples from myeloma subjects will be used in the testing of the methods of the invention.

Primary models: Primary tumors are established from primary multiple myeloma cancers by transferring plasma cells, e.g., CD138 positive cells, isolated from bone marrow aspirates of subjects with multiple myeloma to immunodeficient mice. In order to translate these studies into the clinic, the xenograft models are evaluated with tumor cells from at least two multiple myeloma subjects, preferably diagnosed with differently staged disease. Bone marrow, as a source of multiple myeloma cells, has been used to establish xenograft murine models successfully, see, e.g., Yaccoby et al., 1998, Blood 92:2908-2913, which is incorporated herein by reference in its entirety. These studies will determine the broad range application of the anti-Fc□RIIB antibody in tumor clearance of primary cells. Additionally, the model progression will further characterize the diagnostic and prognostic methods of the present invention. Tumor clearance is tested using anti-CD32B antibody, e.g., h2B6, in CB.17/ICr-SCID mice with adoptively transferred marrow cells.

Heparinized bone marrow aspirates are obtained from subjects with active myeloma during scheduled clinic visits. Samples are separated using ficoll hypaque centrifugation (Histopaque; Pharmacia, Uppsala, Sweden), and the proportion of myeloma cells in the light-density cell fractions is determined using flow cytometric analysis for CD138 expression. Plasma cells may also be sorted and/or purified by FACS based on CD138 expression.

Murine Models. Immunodeficient mice, preferably lacking B and T cell function, e.g., CB.17/ICr-SCID mice, are used for the study. Prior to inoculation of the light-density fract of marrow samples comprising myeloma cells or purified myeloma cells, the mice are exposed to 150 rad X-ray irradiation. Light-density bone marrow cells or myeloma cells are injected directly into a bone of the SCID-hu mice. An increase in the levels of circulating monotypic human Ig (hIg) of the M protein isotype is used as an indicator of myeloma cell growth. After s.c inoculation of tumor cells, mice are followed for clinical and anatomical changes. As needed, mice may be necropsied to correlate total tumor burden with specific organ localization.

Mice will be treated with the selected antibody injected i.p. on day 1, 4, 7, and 15. A starting dose of 4 μg/g of body weight will be used, but additional doses will be tested to establish the relative potency of the antibodies in this model. Standard treatment for myeloma will be used as control (e.g. melphalan, prednisone (MP), thalidomide, dexamethasone (Thal/Dex), vincristine, carmustine, melphalan, cyclophosphamide, prednisone (VBMCP)). Furthermore, potential synergism of combination therapy with any such therapies will be studied. In these studies, tumor growth and morbidity will be monitored to compare antibody treated and control groups. Mice will be sacrificed immediately if moribund or at the completion of the studies. The tumors will then be excised and gross and microscopic necropsy performed. Cytopathology on paraffin-embedded sections and immunohistochemistry on frozen sections will be performed for a morphological and immunological evaluation of the tumor and cellular infiltrates.

6.3 Clinical Trial of Humanized CD32B Binding Agent

A phase I trial of humanized anti-CD32B antibody or agent that binds CD32B administration to subjects with active plasma cell disorder, e.g. multiple myeloma, light-chain amyloidosis, or plasma cell leukemia may be designed to evaluate both effect on disease progression and possible toxicity. Subjects with a suspected plasma cell disorder may be enrolled after positive diagnosis of plasma cell disorder as is well known in the art.

Treatment may be provided as a single therapeutic or in combination with accepted treatments. For example, treatment strategies for multiple myeloma are reviewed in Rajkumar et al., 2002, Mayo Clin. Proc. 77:814, hereby incorporated by reference in its entirety). Recognition of acute or unusual progression of the disease will halt administration of anti-CD32B antibody.

Initial subjects receive 0.5 mg/kg humanized anti-CD32B antibody, e.g., h2B6, administered as a 1 hour intravenous infusion. Given adequate tolerance, the dose will be increased stepwise in subsequent subjects to 5 mg/kg. Additionally, the method for administration may be changed to bolus injection.

Toxicity of humanized antibody therapy is evaluated in subjects according to the World Health Organization Toxicity Criteria: blood pressure, temperature and heart rate are monitored every 10 minutes during mAb infusion, then every hour for 3 hours and finally every 3 hours for 24 hours. Hematologic, renal and liver function tests are conducted every other day for one week and on day 15, 30, 60 and 120 post injection.

Serum and/or tissue samples are obtained once a week for two months so that the effects of mAb tumorigenesis may be determined by methods known in the art, e.g., change serum concentration of M protein, change in hemoglobin value, presence/regression of lytic bone lesions etc. Pathologic studies will assess treatment effect on tissue damage associated with the plasma cell disorder.

### 7. Equivalents

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Various references are cited herein, the disclosure of which are incorporated by reference in their entirety.

1. A method for treating a plasma cell disorder characterized by the expression of CD32B, or ameliorating the symptoms thereof, in a patient in need thereof, comprising:
(a) administering to said patient a therapeutically effective amount of an antibody or fragment thereof that specifically binds CD32B.

2. The method of claim 1, further comprising monitoring the efficacy of said treatment in a patient suffering from said disorder, said method comprising the additional steps of:
b) detecting in a biological sample from said patient a population of cells expressing CD138; and
c) determining the level of expression of CD32B in said cell population, wherein if the level of CD32B expression is equivalent to a control cell population or greater than the CD32B expression from a prior sample from said patient, said treatment is effective.

3. The method of claim 2, wherein steps (b) and (c) are repeated at least once.

4. The method of claim 3, wherein steps (b) and (c) are performed both prior and subsequent to the administration of said antibody or fragment thereof that specifically binds CD32B.

5. The method of claim 1, wherein said treatment is preventing the progression of a plasma cell disorder characterized by the expression of CD32B in a patient in need thereof.

6. The method of claim 1, further comprising monitoring the progression of the plasma cell disorder characterized by the expression of CD32B in a patient suffering therefrom, said method comprising the additional steps of:
b) detecting in a biological sample from said patient a population of cells expressing CD138; and
c) determining the level of expression of CD32B in said cell population wherein if the level of CD32B expression is less than that of a control cell population or less than the CD32B expression from a prior sample from said patient, said disorder is progressing to a more aggressive disease state.

7. The method of claim 1, wherein said antibody or fragment thereof specifically binds CD32B with greater affinity than said antibody specifically binds CD32A.

8. The method of claim 1, wherein said antibody is a murine monoclonal antibody produced by clone 3H7 having ATCC accession number PTA-4592 or a fragment thereof.

9. The method of claim 8, wherein said antibody has been humanized or is a fragment of an antibody that has been humanized.

10. The method of claim 1, wherein said antibody is a murine monoclonal antibody produced by clone 2B6 having ATCC accession number PTA-4591 or a fragment thereof.

11. The method of claim 10, wherein said antibody has been humanized or is a fragment of an antibody that has been humanized.

12. The method of claim 1, wherein said antibody is a human antibody or humanized antibody or fragment thereof.

13. The method of claim 1, wherein said plasma cell disorder is systemic light-chain amyloidosis (AL), multiple myeloma (MM), or plasma cell leukemia (PCL).

14. The method of claim 1, wherein said antibody or fragment thereof modulates at least one activity of CD32B.

15. The method of claim 14, wherein said antibody or fragment thereof agonizes said at least one activity of CD32B.

16. The method of claim 14, wherein said antibody or fragment thereof antagonizes said at least one activity of CD32B.

17. The method of claim 14, wherein said activity is inhibition of B cell receptor mediated signaling.

18. The method of claim 14, wherein said activity is inhibition of cell proliferation.

19. The method of claim 1, wherein said antibody or fragment thereof is conjugated to a therapeutic agent.

20. The method of claim 19, wherein said therapeutic agent is a cytotoxin, a heterologous polypeptide, or an antibody or fragment thereof that immunospecifically binds to a cell surface receptor other than CD32B.

21. The method of claim 20, wherein said cytotoxin is paclitaxel, cytochalasin B, gramicidin D, ethidium bromide, emetine, mitomycin, carmustine, etoposide, tenoposide, vincristine, vinblastine, colchicine, doxorubicin, daunorubicin, dihydroxy anthracin dione, mitoxantrone, mithramycin, melphalan, actinomycin D, 1-dehydrotestosterone, glucocorticoids, procaine, tetracaine, lidocaine, propranolol, puromycin, epirubicin, or cyclophosphamide.

22. The method of claim 1 further comprising administration of one or more additional cancer therapies.

23. The method of claim 22, wherein at least one or said one or more cancer therapies is antibody therapy, cytokine therapy, chemotherapy, hematopoietic stem cell transplantation, B-cell mediated therapy, biological therapy, immunotherapy, radiation therapy, hormonal therapy, or surgery.

24. The method of claim 1, wherein said antibody or fragment thereof comprises one or more amino-acid modification(s) in the Fc domain relative to the wild type Fc domain.

25. The method claim 1, wherein said subject is human.

26. A method for assessing the state of a plasma cell disorder in a subject in need thereof, comprising:
a) detecting in a biological sample from said subject a population of cells expressing CD138; and
b) determining the level of expression of CD32B in said cell population, wherein a variation in the level of CD32B expression from a standard level, a control cell population level, a non-malignant plasma cell population level, or a prior level determined for said subject is associated with the progression, regression, or remission of said plasma cell disorder.

27. The method of claim 26, wherein said assessment of the state of a plasma cell disorder is assessing the aggressivity of a plasma cell disorder in a subject having said disorder, additionally comprising the step of:
c) comparing the level of CD32B expression determined in step (b) to that of a control cell population or to a standard level,
wherein the ratio of the level determined in (b) to that of a control cell population or to a standard level is inversely proportional to the aggressivity of said disorder.

28. The method of claim 26, wherein said assessment of the state of a plasma cell disorder is monitoring the progression of a plasma cell disorder in a patient diagnosed with said disorder, additionally comprising the steps of:
c) repeating steps (a) and (b) at least once at a subsequent point in time; and
d) comparing the at least one level determined in step (c) with the level determined in step (b).

29. The method of claim 28, wherein the change of CD32B expression associated with the progression of said disorder is a decrease in CD32B expression.

30. The method of claim 28, wherein the change in CD32B expression associated with the regression or remission of said disorder is an increase in CD32B expression.

31. The method of claim 28, wherein, prior to step (c), one or more cancer therapies are administered to said subject.

32. The method of claim 26, wherein said plasma cell disorder is systemic light-chain amyloidosis (AL), multiple myeloma (MM), or plasma cell leukemia (PCL).

33. The method of claim 26, wherein said assessment of the state of a plasma cell disorder is diagnosis of multiple myeloma.

34. The method of claim 26, wherein the population of cells expressing CD138 is detected by an additional method comprising:
(a) contacting said sample with an effective amount of an antibody or fragment thereof that immunospecifically binds CD138 and comprises a detectable marker; and
(b) detecting the binding of said antibody or fragment thereof.

35. The method of claim 34, wherein said detectable marker is a chemiluminescent, enzymatic, fluorescent, or radioactive label.

36. The method of claim 34, wherein said detectable marker is fluorescent and detection is by fluorescence activated cell sorting (FACS) or flow cytometric (FCM) analysis.

37. The method of claim 26, wherein the level of CD32B expression is determined by an additional method comprising:
(a) contacting said sample with an effective amount of an antibody or fragment thereof that immunospecifically binds CD32B and comprises a detectable marker; and
(b) determining the level of binding of said antibody or fragment thereof.

38. The method of claim 37, wherein said detectable marker is a chemiluminescent, enzymatic, fluorescent, or radioactive label.

39. The method of claim 37, wherein said detectable marker is fluorescent and detection is by fluorescence activated cell sorting (FACS) or flow cytometric (FCM) analysis.

40. The method of claim 37, wherein said antibody or fragment thereof that immunospecifically binds CD32B is a murine monoclonal antibody produced by clone 2B6 having ATCC accession number PTA-4591 or by clone 3H7 having ATCC accession number PTA-4592.

41. The method of claim 37, wherein said antibody or fragment thereof competes for binding with a murine monoclonal antibody produced by clone 2B6 having ATCC accession number PTA-4591 or by clone 3H7 having ATCC accession number PTA-4592.

42. The method of claim 26, wherein the population of cells expressing CD138 and the level of CD32B expression is determined by an additional method comprising:
(a) contacting said sample with an effective amount of a first antibody or fragment thereof that immunospecifically binds CD138 and comprises a first detectable marker;
(b) contacting said sample with an effective amount of a second antibody or fragment thereof that immunospecifically binds CD32B and comprises a second detectable marker; and
(c) concurrently detecting the binding of said first antibody or fragment thereof and determining the level of binding of said antibody or fragment thereof.

43. The method of claim 42, wherein said first detectable marker or said second detectable marker, or both is a chemiluminescent, enzymatic, fluorescent, or radioactive label.

44. The method of claim 42, wherein said first detectable marker or said second detectable marker, or both, is fluorescent and detection is by fluorescence activated cell sorting (FACS) or flow cytometric (FCM) analysis.

45. The method of claim 42, wherein said antibody or fragment thereof that immunospecifically binds CD32B is a murine monoclonal antibody produced by clone 2B6 having ATCC accession number PTA-4591 or by clone 3H7 having ATCC accession number PTA-4592.

46. The method of claim 42, wherein said antibody or fragment thereof competes for binding with a murine monoclonal antibody produced by clone 2B6 having ATCC accession number PTA-4591 or by clone 3H7 having ATCC accession number PTA-4592.

47. The method of claim 26, wherein, prior to step (b), said cell population is isolated from said sample.

48. The method of claim 43, wherein said detectable marker is a fluorescent label and said cell population is isolated by fluorescence activated cell sorting (FACS).
